SEQUENCE LISTING (5 sequences)

SEQ ID NO: 1 - DNA, 1215 bp, synthetic construct
  source 1..1215: mol_type = other DNA
  CDS 1..1215: note = GS2-IpaH9.8deltaLRR
atggtttcttctgttccgaccaaactggaagttgttgctgcgaccccgactagcctgctgatcagctgggatgctcctgcagttaccgtcgatcattactatatcacgtacggtgaaaccggtcattactggtactaccaggcattcgcggtacctggttccaagtctactgctaccatcagcggcctgagcccgggtgtcgactataccatcactgtatacgcaccgttctctgttccggttatgagcccaatctcgattaactaccgtaccgctagcctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggatattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaaatatgagatgctggagaatgagtaccctcagagggtggctgaccggctgaaagcatcaggtctgagcggtgatgcggatgcggagagggaagccggtgcacaggtgatgcgtgagactgaacagcagatttaccgtcagctgactgacgaggtactggccctgcgattgtctgaaaacggctcacaactgcaccattcacctgcaggagcagactacaaggacgatgacgacaagggacatcatcatcatcatcactaa

SEQ ID NO: 2 - DNA, 1182 bp, synthetic construct
  source 1..1182: mol_type = other DNA
  CDS 1..1182: note = GS2-IpaH9.8deltaLRR (K-free)
atggtttcttctgttccgaccgcactggaagttgttgctgcgaccccgactagcctgctgatcagctgggatgctcctgcagttaccgtcgatcattactatatcacgtacggtgaaaccggtcattactggtactaccaggcattcgcggtacctggttccgcgtctactgctaccatcagcggcctgagcccgggtgtcgactataccatcactgtatacgcaccgttctctgttccggttatgagcccaatctcgattaactaccgtaccgctagcctggctgatgccgtgacagcatggttcccggaaaacgcacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaagcactcagtgcctctgcggagcttcgacagcagtctttcgctgttgctgctgatgccactgagagctgtgaggaccgtgtcgcgctcacatggaacaatctccgggcaaccctcctggtccatcaggcatcagaaggccttttcgataatgataccggcgctctgctctccctgggcagggaaatgttccgcctcgaaattctggaggatattgcccgggatgcagtcagaactctccattttgtggatgagatagaagtctacctggccttccagaccatgctcgcagaggcacttcagctctccactgccgtggcggaaatgcgtttctatggcgtgtcgggagtgacagcaaatgacctccgcactgccgaagccatggtcagaagccgtgaagagaatgaatttacggactggttctccctctggggaccatggcatgctgtactggcgcgtacggaagctgaccgctgggcgcaggcagaagagcaggcatatgagatgctggagaatgagtaccctcagagggtggctgaccggctggcagcatcaggtctgagcggtgatgcggatgcggagagggaagccggtgcacaggtgatgcgtgagactgaacagcagatttaccgtcagctgactgacgaggtactggccctgcgattgtctgaaaacggctcacaactgcaccattcacctgcacatcatcatcatcatcactaa

SEQ ID NO: 3 - DNA, 1182 bp, synthetic construct
  source 1..1182: mol_type = other DNA
  CDS 1..1182: note = GS2-IpaH9.8deltaLRR (K-free/ C337A)
atggtttcttctgttccgaccgcactggaagttgttgctgcgaccccgactagcctgctgatcagctgggatgctcctgcagttaccgtcgatcattactatatcacgtacggtgaaaccggtcattactggtactaccaggcattcgcggtacctggttccgcgtctactgctaccatcagcggcctgagcccgggtgtcgactataccatcactgtatacgcaccgttctctgttccggttatgagcccaatctcgattaactaccgtaccgctagcctggctgatgccgtgacagcatggttcccggaaaacgcacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaagcactcagtgcctctgcggagcttcgacagcagtctttcgctgttgctgctgatgccactgagagcgctgaggaccgtgtcgcgctcacatggaacaatctccgggcaaccctcctggtccatcaggcatcagaaggccttttcgataatgataccggcgctctgctctccctgggcagggaaatgttccgcctcgaaattctggaggatattgcccgggatgcagtcagaactctccattttgtggatgagatagaagtctacctggccttccagaccatgctcgcagaggcacttcagctctccactgccgtggcggaaatgcgtttctatggcgtgtcgggagtgacagcaaatgacctccgcactgccgaagccatggtcagaagccgtgaagagaatgaatttacggactggttctccctctggggaccatggcatgctgtactggcgcgtacggaagctgaccgctgggcgcaggcagaagagcaggcatatgagatgctggagaatgagtaccctcagagggtggctgaccggctggcagcatcaggtctgagcggtgatgcggatgcggagagggaagccggtgcacaggtgatgcgtgagactgaacagcagatttaccgtcagctgactgacgaggtactggccctgcgattgtctgaaaacggctcacaactgcaccattcacctgcacatcatcatcatcatcactaa

SEQ ID NO: 4 - DNA, 657 bp, synthetic construct
  source 1..657: mol_type = other DNA
  CDS 1..657: note = D16-CHIPdeltaTPR
atgaggaccctgaacgagatctaccactggttcaccaggatgttcgccttcttcggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggacatcatcatcatcatcactaa

SEQ ID NO: 5 - DNA, 657 bp, synthetic construct
  source 1..657: mol_type = other DNA
  CDS 1..657: note = D16-CHIPdeltaTPR (K-free)
atgaggaccctgaacgagatctaccactggttcaccaggatgttcgccttcttcggcagcggttctggccggctgaacttcggggacgacatccccagcgctcttcgaatcgcgagaagaagacgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccagacacgacagatacatggcggacatggacgagcttttttctcaggtggatgagagaaggagaagacgagacatccccgactacctgtgtggcagaatcagctttgagctgatgcgggagccgtgcatcacgcccagtggcatcacctacgaccgcagagacatcgaggagcacctgcagcgtgtgggtcattttgaccccgtgacccggagccccctgacccaggaacagctcatccccaacttggctatgagagaggttattgacgcattcatctctgagaatggctgggtggaggattacgtcgacggagcagactacaaggacgatgacgacaagggacatcatcatcatcatcactaa

(19) United States
(12) Patent Application Publication
(10) Pub. No.: US 2026/0250647 A1
(43) Pub. Date: Aug. 27, 2026

(54) LYSINE-FREE UBIQUIBODY VARIANTS FOR LONG-LIVED INTRACELLULAR PROTEIN SILENCING

(71) Applicant: CORNELL UNIVERSITY, Ithaca, NY (US)

(72) Inventors: Matthew DELISA, Ithaca, NY (US); Morgan LUDWICKI, Ithaca, NY (US); Tianzheng YE, Ithaca, NY (US); Connor MONTICELLO, Ithaca, NY (US)

(21) Appl. No.: 18/845,621

(22) PCT Filed: Mar. 10, 2023
(86) PCT No.: PCT/US2023/064149

§ 371 Date: Sep. 10, 2024

Related U.S. Application Data
(60) Provisional application No. 63/318,610, filed on Mar. 10, 2022.

(51) Int. Cl. C12N 9/10; C07K 16/18; C07K 16/24; C07K 16/28; C07K 16/30; C07K 16/40; G01N 33/50; G01N 33/575

(52) U.S. Cl. CPC C12N 9/104; C07K 16/18; C07K 16/241; C07K 16/2839; C07K 16/2851; C07K 16/2863; C07K 16/2878; C07K 16/3007; C07K 16/3092; C07K 16/40; G01N 33/5011; G01N 33/5758; C07K 2319/30; G01N 2333/9108; G01N 2500/20

(57) ABSTRACT

The present disclosure relates to a chimeric protein molecule comprising a degradation domain including an E3 ubiquitin ligase motif without lysine residues and a targeting domain comprising a substrate-binding motif which is heterologous to the E3 ubiquitin ligase motif. A linker couples the degradation domain to the targeting domain. Also disclosed are compositions as well as methods of treating a disease, substrate silencing, forming a ribonucleoprotein, screening agents for therapeutic efficacy against a disease, and methods of screening for disease biomarkers, as well as mRNA molecules, vectors, and encapsulated nucleic acid molecules encoding chimeric protein molecules.

This application claims the priority benefit of U.S. Provisional Patent Application Ser. No. 63/318,610, filed Mar. 10, 2022, which is hereby incorporated by reference in its entirety.

### FIELD

The present disclosure relates to targeted protein silencing using chimeras between antibodies and E3 ubiquitin ligase motifs without lysine residues.

### SEQUENCE LISTING

The Sequence Listing has been submitted electronically in XML format (“Sequence Listing XML”) and is hereby incorporated by reference in its entirety. Said XML copy, created on Mar. 10, 2023, is named 147402-009131-Sequence-Listing and is 12,171 bytes in size.

### BACKGROUND

Following discovery of the ubiquitin-proteasome system (“UPS”) in the 1980's, researchers have exploited its capacity to function as a degradative regulator of protein profusion. The UPS ensures homeostatic concentrations of intracellular proteins via the proteasome, which recognizes and degrades ubiquitinated proteins. The process of ubiquitination involves a cascade of three enzymes, i.e., the ubiquitin activating enzyme (“E1”), the ubiquitin conjugating enzyme (“E2”), and the ubiquitin ligase (“E3”). In brief, target protein modification, through ubiquitination, occurs when ubiquitin is activated by E1, which then transfers the highly conserved ubiquitin molecules to an E2 intermediate. Finally, E3-mediated ligation permits the covalent attachment of ubiquitin to a substrate. Typically, ubiquitin-chain elongation occurs pursuant to the concerted action of E2 and E3, albeit through a process that is not completely understood (Hochstrasser, M., “Lingering Mysteries of Ubiquitin-Chain Assembly.” Cell 124(1): 27-34 (2006)).

Ubiquitination imparts a conduit system for elucidating dynamic protein interactions by manipulating the cellular machinery, i.e., E1, E2, and E3. Previously, however, cellular characteristics could only be studied by employing, for example, genetic “knock-outs” or RNA interference (“RNAi”) technology. Notwithstanding the benefits of such technology, systems that function at the genetic level fail to provide phenotypic insight into cellular processes and disease etiology. While modified ubiquitin E3 enzymes have been generated, these ligases are circumscribed insofar as they possess native substrate specificity.

The first successful in vivo redirection of a ubiquitin bound substrate—to the proteasome—was performed by engineering a multimeric E3 protein complex viz SCF (Skp1, Cullin, F box-containing proteins) in yeast. Zhou, et al., “Harnessing the Ubiquitination Machinery to Target the Degradation of Specific Cellular Proteins.” Mol. Cell 6(3): 751-756 (2000) (“Zhou 2000”) demonstrated that by generating chimeric proteins possessing a target protein binding partner, the F-box containing protein Cdc4p—which functions as a substrate recognition complex—could redirect the SCF complex to degrade the target protein. Furthermore, it was shown that the human homolog of Cdc4p, i.e., βTrCP, could be engineered to selectively degrade hypophosphorylated forms of target proteins. See Zhou (2000); Zhang et al. “Exploring the Functional Complexity of Cellular Proteins by Protein Knockout.” Proc. Natl. Acad. Sci. USA 100(24): 14127-14132 (2003); and Zhou et al. “Targeted Protein Degradation.” Curr. Opin. Chem. Biol. 9(1): 51-45 (2005) (“Zhou 2005”).

Nevertheless, it was determined that the core SCF complex was encumbered by the overexpression of F-box chimeras, which therefore curtailed the ubiquitination of both native and novel substrates. See Zhou (2005). A simplified E3 chimera was created by utilizing a monomeric U-box ubiquitin ligase and fusing it to a known substrate, i.e., c-Myc, a proto-oncogene transcription factor. See Hatakeyama et al., “Targeted Destruction of c-Myc by an Engineered Ubiquitin Ligase Suppresses Cell Transformation and Tumor Formation.” Cancer Res. 65(17): 7874-7879 (2005). In each of these cases, however, pre-existing interactions with native proteins were required to facilitate degradation.

Moreover, it has been shown that the configuration of F-box proteins exclude the possibility of fusion with antibody single-chain fragments (“scFv”) for use in targeted degradation. See Melchionna et al., “A Protein Silencing Switch by Ligand-induced Proteasome-targeting Intrabodies.” J. Mol. Biol. 374, 641-654 (2007). Consequently, a facile approach for effective post-translational protein silencing remains an important consideration in the development of new strategies for elucidating novel molecular mechanisms, drug targets, therapeutic and prognostic determinations of disease, and research-based applications coterminous with the same.

The present disclosure is directed to overcoming these and other deficiencies in the art.

### SUMMARY

One aspect of the present disclosure relates to a chimeric protein molecule comprising (i) a degradation domain including an E3 ubiquitin ligase motif without lysine residues and (ii) a targeting domain comprising a substrate-binding motif which is heterologous to the E3 ubiquitin ligase motif. A linker couples the degradation domain to the targeting domain. In some embodiments, the substrate-binding motif and/or the linker has no lysine residues. In some embodiments, the substrate is a protein substrate.

A further aspect of the present disclosure relates to a composition comprising the chimeric protein molecule and a pharmaceutically-acceptable carrier.

Another aspect of the present disclosure relates to a method of treating a disease. This method involves administering a composition according to the present disclosure to a subject having a disease, where the subject to whom the composition is administered has an increased expression level of a substrate (e.g., a protein substrate) compared to a subject not afflicted with the disease.

Another aspect of the present disclosure relates to a method for protein substrate silencing. This method involves selecting a protein substrate to be silenced and providing a chimeric protein molecule according to the present disclosure. This method further involves contacting the protein substrate and the chimeric protein molecule under conditions effective to permit the formation of a protein substrate-molecule complex, where the complex mediates degradation of the protein substrate to be silenced.

Another aspect of the present disclosure relates to forming a ribonucleoprotein. This method involves providing a mRNA encoding the chimeric protein molecule according to the present disclosure and providing one or more polyadenosine binding proteins (“PABP”). This method further involves assembling a ribonucleoprotein complex from the mRNA and the one or more PABPs. In some embodiments, the chimeric protein molecule is an isolated chimeric protein molecule.

Another method of the present disclosure relates to a method of screening agents for therapeutic efficacy against a disease. This method involves providing a biomolecule whose presence is mediated by a disease state. A test agent comprising (i) a degradation domain including an E3 ubiquitin ligase motif without lysine residues, (ii) a targeting domain comprising a substrate-binding motif which is heterologous to the E3 ubiquitin ligase motif, and (iii) a linker coupling the degradation domain to the targeting domain are provided. The biomolecule and the test agent are contacted under conditions effective for the test agent to facilitate degradation of the biomolecule. The level of the biomolecule, as a result of the contacting, is determined and the test agent which, based on the determining, decreases the level of the biomolecule is identified as being a candidate for therapeutic efficacy against the disease.

Another aspect of the present disclosure relates to a method of screening for disease biomarkers. This method involves providing a sample of diseased cells expressing one or more ligands. A plurality of chimeric protein molecules comprising (i) a degradation domain including an E3 ubiquitin ligase motif without lysine residues, (ii) a targeting domain comprising a substrate-binding motif which is heterologous to the E3 ubiquitin ligase motif, and (iii) a linker coupling the degradation domain to the targeting domain are provided. This method further involves contacting the sample with the plurality of chimeric protein molecules under conditions effective for the diseased cells to fail to proliferate in the absence of the chimeric protein molecule, determining which of the chimeric protein molecules permit the diseased cells to proliferate, and identifying, as biomarkers for the disease, based on the determining the ligands which bind to the chimeric protein molecules and permit diseased cells to proliferate.

Another aspect of the present disclosure relates to an mRNA molecule encoding a chimeric protein molecule according to the present disclosure.

A further aspect of the present disclosure relates to a vector encoding an mRNA molecule according to the present disclosure.

A further aspect of the present disclosure relates to an encapsulated nucleic acid molecule comprising: (i) a mRNA molecule according to the present disclosure or a vector according to the present disclosure and (ii) a protein and/or polymer complex.

The present disclosure provides methods and compositions for the creation of ubiquibodies—engineered chimeras between a synthetic binding protein (e.g., antibodies, DARPins, FN3, monobodies, nanobodies, etc.) and an E3 ubiquitin ligase—that have extended half-life inside of cells. A limitation of the previously invented ubiquibodies is their susceptibility to autodegradation, which is caused by the E3 ligase's natural propensity for promoting self-conjugation of ubiquitin molecules onto internal lysine residues that are present in either the E3 ligase domain or the synthetic binding domain, or both, of the ubiquibody. To overcome this limitation, “lysine-free” ubiquibodies in which all internal lysine residues of the E3 ligase domain and the synthetic binding domain of the ubiquibody are removed by site-directed mutagenesis have been engineered. The examples of the present disclosure demonstrate that eliminating all of the possible sites for autoubiquitination results in lysine-free ubiquibodies that are resistant to proteasomal degradation without compromising the ability to degrade their targets. Surprisingly, lysine-free ubiquibody variants catalyze targeted protein degradation to an extent that is as good or better than original parental ubiquibodies. Importantly, by side-stepping this key elimination mechanism, the resulting lysine-free ubiquibody variants exhibited increased intracellular half-life and longer duration of action relative to their parental ubiquibody sequences following delivery into host cells. The present disclosure therefore paves the way for therapeutic deployment of highly durable ubiquibodies that achieve long durations of action through extended intracellular half-life.

### BRIEF DESCRIPTION OF THE DRAWINGS

FIG. 1 shows the structure of IpaH9.8 with lysine residues highlighted. Structure of IpaH9.8 catalytic domain lacking its native substrate-binding domain (IpaH9.8ΔLRR). This catalytic domain used in ubiquibodies contains 9 lysine residues (note that K420 is hidden and thus not depicted). The GS2 synthetic binding domain has 2 lysine residues (not shown).

FIG. 2 is a bar graph showing flow cytometric quantification of EGFP fluorescence activity in HEK293-T cells with no plasmid (lane 1) or transiently transfected with the following plasmids: pcDNA3-EGFP alone (lane 2), pcDNA3-EGFP together with a plasmid encoding a GFP-specific ubiquibody, namely GS2-uAb (lane 3), pcDNA3-EGFP together with a plasmid encoding the same GFP-specific ubiquibody but with all lysines mutated to alanine, namely GS2-uAb(K-free)(lane 4), or pcDNA3-EGFP together with a plasmid encoding the catalytically inactive lysine-free ubiquibody, namely GS2-uAb(K-free/C337A)(lane 5). Fluorescence intensity was measured 48 hours post transfection and normalized to the fluorescence measured in HEK293-T cells carrying pcDNA3-EGFP only (lane 2). Data are the average of three biological replicates and error is the standard error of the mean. The results indicate that the lysine-free ubiquibody, GS2-uAb(K-free) degraded EGFP more efficiently than the parental ubiquibody, GS2-uAb, that contains the full complement of lysine residues.

FIG. 3 is a blot demonstrating that lysine-free ubiquibodies are well expressed in mammalian cells. Expression of a lysine-free ubiquibody, namely GS2-uAb(K-free), or the catalytic mutant of the lysine-free ubiquibody, namely GS2-uAb(K-free/C337A), in HEK293-T cell lysates. Expression was evaluated by Western blot analysis using an anti-polyhistidine antibody (α-His). K-free ubiquibodies have all lysine residues in the GS2 domain (2 total) and in the IpaH9.8ΔLRR catalytic domain (9-total) substituted with alanine. Western blot confirms that substitution of all lysine residues is well tolerated in terms of soluble intracellular expression of the ubiquibody chimeras. Molecular weight (MW) ladder indicated at left. Expected MW of each lysine-free ubiquibody is approximately 48 kDa.

FIG. 4 are blots demonstrating that lysine-free ubiquibodies evade ubiquitination in mammalian cells. Ubiquitination of a lysine-free ubiquibody, namely GS2-uAb(K-free), or the catalytic mutant of the lysine-free ubiquibody, namely GS2-uAb (K-free/C337A), in HEK293-Tcells. The extent of ubiquitination was evaluated by Western blot analysis using an anti-His antibody against the ubiquibody (left blot) and an anti-ubiquitin antibody against the conjugated ubiquitin (right blot). Western blots confirm that substitution of all lysine residues results in complete elimination of autoubiquitination of the K-free ubiquibody chimera (lane 3), whereas the IpaH9.8 catalytic domain alone or the original ubiquibody, each with full complement of lysines, undergo extensive ubiquitination (lanes 1 and 2). Molecular weight (MW) ladder indicated at left.

FIG. 5 is a schematic of the structure of human CHIP catalytic domain lacking its native substrate-binding domain (CHIPΔTPR) with all lysine residues highlighted. This catalytic domain used in ubiquibodies contains 11 lysine residues.

FIG. 6 is a bar graph showing flow cytometric quantification of FOXP3-GFP fluorescence activity in HEK293-T cells with no plasmid (lane 1) or transiently transfected with the following plasmids: pcDNA3-FOXP3-GFP alone (lane 2), pcDNA3-FOXP3-GFP together with a plasmid encoding the FOXP3-specific ubiquibody, namely D16-uAb (lane 3), or pcDNA3-FOXP3-GFP together with a plasmid encoding the same FOXP3-specific ubiquibody but with all lysines mutated to arginine, namely D16-uAb(K-free) (lane 4). Fluorescence intensity was measured 48 hours post transfection and normalized to the fluorescence measured in HEK293-T cells carrying pcDNA3-FOXP3-GFP only (lane 2). Data are the average of three biological replicates and error is the standard error of the mean. The results indicate that the lysine-free ubiquibody, D16-uAb(K-free) degraded FOXP3-GFP more efficiently than the parental ubiquibody, D16-uAb.

### DETAILED DESCRIPTION

It is to be appreciated that certain aspects, modes, embodiments, variations, and features of the present disclosure are described below in various levels of detail to provide a substantial understanding of the present technology. The definitions of certain terms as used in this specification are provided below. Unless defined otherwise, all technical and scientific terms used herein generally have the same meaning as commonly understood by one of ordinary skill in the art to which the subject matter of the present disclosure belongs.

In practicing the subject matter of the present disclosure, many conventional techniques in molecular biology, protein biochemistry, cell biology, immunology, microbiology and recombinant DNA may be used. These techniques are well-known and are explained in, e.g., Current Protocols in Molecular Biology, Vols. I-III Ausubel, Ed. (1997); Sambrook et al., Molecular Cloning. A Laboratory Manual, Second Ed. (Cold Spring Harbor Laboratory Press, Cold Spring Harbor, N.Y. (1989)); DNA Cloning. A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, New York (1987)); and Meth. Enzymol., Vols. 154 and 155, Wu & Grossman, and Wu, Eds., respectively, which are hereby incorporated by reference in their entirety. Methods to detect and measure levels of polypeptide gene expression products, i.e., gene translation level, are well-known in the art and include the use polypeptide detection methods such as antibody detection and quantification techniques. See also, Strachan & Read, Human Molecular Genetics, Second Edition. (John Wiley and Sons, Inc., New York (1999), which is hereby incorporated by reference in its entirety.

As used herein, the term “amino acid” includes naturally-occurring amino acids, L-amino acids, D-amino acids, and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, e.g., an α-carbon that is bound to a hydrogen, a carboxyl group, an amino group, and an R group, e.g., homoserine, norleucine, methionine sulfoxide, methionine methyl sulfonium. Such analogs have modified R-groups, e.g., norleucine, or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein the term “antibody” refers to an immunoglobulin and any antigen-binding portion of an immunoglobulin, e.g., IgG, IgD, IgA, IgM and IgE, or a polypeptide that contains an antigen binding site, which specifically or “immunospecifically binds” to, or “immunoreacts with”, an immunogen, antigen, substrate, and the like. Antibodies can comprise at least one heavy (H) chain and at least one light (L) chain inter-connected by at least one disulfide bond. The term “V_{H}” refers to a heavy chain variable region of an antibody. The term “V_{L}” refers to a light chain variable region of an antibody. In some embodiments, the term “antibody” specifically covers monoclonal and polyclonal antibodies. A “polyclonal antibody” refers to an antibody which has been derived from the sera of animals immunized with an antigen or antigens. A “monoclonal antibody” refers to an antibody produced by a single clone of hybridoma cells.

Antibody-related molecules, domains, fragments, portions, etc., useful as targeting domains of the disclosure include, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341:544-546, (1989)), which consists of a V_{H} domain; and (vi) an isolated complementary determining region (CDR). As such “antibody fragments” can comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. Single-chain antibody molecules may comprise a polymer with a number of individual molecules, for example, dimer, trimer or other polymers.

As used herein, the terms “biomarker” or “biomolecule” or “molecule” refer to a polypeptide (of a particular expression level) which is differentially present in a sample taken from patients having a disease as compared to a comparable sample taken from a control subject or a population of control subjects.

As used herein, the terms “effective amount” or “therapeutically effective amount” of a chimeric protein molecule or composition is a quantity sufficient to achieve a desired therapeutic and/or prophylactic effect, for example, an amount which results in the prevention of or a decrease in the symptoms associated with a disease that is being treated. The amount of compound administered to the subject will depend on the type and severity of the disease and on the characteristics of the individual, such as general health, age, sex, body weight and tolerance to drugs. It will also depend on the degree, severity or stage of disease. The skilled artisan will be able to determine appropriate dosages depending on these and other factors.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. Typically, an epitope will be a determinant region form a substrate, which can be recognized by one or more targeting domains.

To screen for targeting domains or substrates which possess an epitope, a routine cross-blocking assay such as that described in Antibodies, A Laboratory Manual, Cold Spring Harbor Laboratory, Ed Harlow and David Lane (1988), which is hereby incorporated by reference in its entirety, can be performed. This assay can be used to determine if a targeting domain binds the same site or epitope of a substrate as a different targeting domain, antibody, antibody fragment and the like. Alternatively, or additionally, epitope mapping can be performed by methods known in the art. For example, the antibody sequence can be mutagenized such as by alanine scanning, to identify contact residues. In a different method, peptides corresponding to different regions of substrate can be used in competition assays with a test target domain or with a test antibody and a target domain or an antibody with a characterized epitope.

As used herein, the term “hypervariable region” refers to the amino acid residues of an antibody which are responsible for antigen-binding. The hypervariable region generally comprises amino acid residues from a “complementarity determining region” or “CDR”, e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991), which is hereby incorporated by reference in its entirety, and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H}(Chothia and Lesk J. Mol. Biol. 196:901-917 (1987)), which is hereby incorporated by reference in its entirety.

As used herein, the terms “ligand” or “substrate” refer to substances that are able to bind to and form transient or stable complexes with a protein, molecule, chimeric protein molecule, ligand (dimer), substrate (dimer), a second substrate, a second ligand, target domain, regions, portions, and fragments thereof, ubiquitin or U-box motif regions, domains, or portions thereof, biomolecules, biomarkers, and the like, to serve a biological purpose, for example a substrate which interacts with an enzyme in the process of an enzymatic reaction. Ligands also include signal triggering molecules which bind to sites on a target protein, by intermolecular forces such as ionic bonds, hydrogen bonds and Van der Waals forces. In some embodiments, substrates bind ligands and/or ligands bind substrates.

As used herein, the terms “modification(s)” or “amino acid modification” of a polypeptide, protein, region, domain, or the like, refers to a change in the native sequence such as a deletion, addition, or substation of a desired residue. Such modified polypeptides are prepared by introducing appropriate nucleotide changes into the antibody nucleic acid, or by peptide synthesis. Any combination of deletion, insertion, and substitution is made to obtain the antibody of interest, as long as the obtained antibody possesses the desired properties. The modification also includes the change of the pattern of glycosylation of the protein. A useful method for identification of preferred locations for mutagenesis is called “alanine scanning mutagenesis” as described by Cunningham and Wells in Science, 244:1081-1085 (1989), which is hereby incorporated by reference in its entirety. The mutated antibody is then screened for the desired activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Nevertheless, the monoclonal antibodies to be used in accordance with the present disclosure may be made by the hybridoma method first described by Kohler et al., Nature 256:495 (1975), which is hereby incorporated by reference in its entirety, or may be made by recombinant DNA methods (see, e.g., U.S. Pat. No. 4,816,567, which is hereby incorporated by reference in its entirety). The “monoclonal antibodies” may also be isolated from phage antibody libraries using the techniques described in Clackson et al., Nature 352:624-628 (1991) and Marks et al., J. Mol. Biol. 222:581-597 (1991), for example, which are hereby incorporated by reference in their entirety.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

As used herein, the terms “reference level” or “control level” refer to an amount or concentration of biomarker (or biomolecule, ligand, substrate and the like) which may be of interest for comparative purposes. In some embodiments, a reference level may be the level of at least one biomarker expressed as an average of the level of at least one biomarker taken from a control population of healthy subjects or from a diseased population possessing aberrant expression of a protein or substrate. In another embodiment, the reference level may be the level of at least one biomarker in the same subject at an earlier time, i.e., before the present assay. In even another embodiment, the reference level may be the level of at least one biomarker in the subject prior to receiving a treatment regime.

As used herein, the term “sample” may include, but is not limited to, bodily tissue or a bodily fluid such as blood (or a fraction of blood such as plasma or serum), lymph, mucus, tears, saliva, sputum, urine, semen, stool, CSF, ascities fluid, or whole blood, and including biopsy samples of body tissue. A sample may also include an in vitro culture of microorganisms grown from a sample from a subject. A sample may be obtained from any subject, e.g., a subject/patient having or suspected to have a disease or condition characterized by a disease.

As used herein, the term “screening” means determining whether a chimeric protein molecule or composition has capabilities or characteristics of preventing or slowing down (lessening) the targeted pathologic condition stated herein, namely a disease or condition characterized by defects in specified disease.

As used herein, the terms “single chain antibodies” or “single chain Fv (scFv)” refer to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). See, e.g., Bird et al., Science 242:423-426 (1988) and Huston et al., Proc. Natl. Acad. Sci. USA 85:5879-5883 (1988), which is hereby incorporated by reference in its entirety. Such single chain antibodies are included by reference to the term “antibody” fragments, and can be prepared by recombinant techniques or enzymatic or chemical cleavage of intact antibodies.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a disease or condition.

As used herein, the term “variable” refers to the fact that certain segments of the variable domains differ extensively in sequence among antibodies. The V domain mediates antigen binding and defines specificity of a particular antibody for its particular antigen. However, the variability is not evenly distributed across the amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies. See Kabat et al., “Sequences of Proteins of Immunological Interest,” 5th Ed. Public Health Service, National Institutes of Health, Bethesda, Md. (1991), which is hereby incorporated by reference in its entirety). The constant domains are not involved directly in binding an antibody to an antigen, but exhibit various effector functions, such as participation of the antibody in antibody dependent cellular cytotoxicity (ADCC).

As used herein, the terms “variant” or “mutant” are used to refer to a protein or peptide which differs from a naturally occurring protein or peptide, i.e., the “prototype” or “wild-type” protein, by modifications to the naturally occurring protein or peptide, but which maintains the basic protein and side chain structure of the naturally occurring form. Such changes include, but are not limited to: changes in one, few, or even several amino acid side chains; changes in one, few or several amino acids, including deletions, e.g., a truncated version of the protein or peptide, insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A “variant” or “mutant” can have enhanced, decreased, changed, or substantially similar properties as compared to the naturally occurring protein or peptide.

As used herein, the term “ubiquitination” refers to the attachment of the protein ubiquitin to lysine residues of other molecules. Ubiquitination of a molecule, such as a peptide or protein, can act as a signal for its rapid cellular degradation, and for targeting to the proteasome complex.

As used herein, the terms “chimeric protein molecule” or “ubiquibody” are used interchangeably and refer to a molecule possessing a degradation domain and a targeting domain, attached by a linker region, as defined herein.

Accordingly, one aspect of the present disclosure relates to a chimeric protein molecule comprising (i) a degradation domain including an E3 ubiquitin ligase motif without lysine residues and (ii) a targeting domain comprising a substrate-binding motif which is heterologous to the E3 ubiquitin ligase motif. A linker couples the degradation domain to the targeting domain.

In some embodiments of the compositions and methods according to the present disclosure, the chimeric protein molecule (or test agent) is an isolated chimeric protein molecule (or isolated test agent). As used herein, the terms “isolated” or “purified” polypeptide, peptide, molecule, or chimeric protein molecule, is substantially free of cellular material or other contaminating polypeptides from the cell or tissue source from which the agent is derived, or substantially free from chemical precursors or other chemicals when chemically synthesized. For example, a chimeric protein molecule would be free of materials that would interfere with such a molecule's intended function, diagnostic or therapeutic uses. Such interfering materials may include proteins or fragments other than the materials encompassed by the chimeric protein molecule, enzymes, hormones and other proteinaceous and nonproteinaceous solutes.

In some embodiments of the compositions and methods according to the present disclosure, the linker is heterologous to the degradation domain and the targeting domain. In accordance with such embodiments, the linker is heterologous to both the E3 ubiquitin ligase motif of the degradation domain and the substrate-binding motif of the targeting domain.

As described herein, the substrate-binding motif of the targeting domain is heterologous to the E3 ubiquitin ligase motif of the degradation domain. Accordingly, the degradation domain may be heterologous to the targeting domain. Likewise, in some embodiments, the degradation domain does not comprise a substrate-binding motif. Similarly, in certain embodiments, the targeting domain does not comprise a ubiquitin ligase motif (e.g., an E3 ubiquitin ligase motif).

In some embodiments of the compositions and methods according to the present disclosure, the E3 ubiquitin ligase motif is a variant E3 ubiquitin ligase motif. As described herein supra, the term “variant” refers to a protein or peptide which differs from a naturally occurring protein or peptide, i.e., the “prototype” or “wild-type” protein, by modifications to the naturally occurring protein or peptide, but which maintains the basic protein and side chain structure of the naturally occurring form. In accordance with the compositions and methods according to the present disclosure, the E3 ubiquitin ligase motif is a variant E3 ubiquitin ligase motif which has been modified to remove any lysine residues present in its “prototype”, “wild-type”, or “naturally-occurring” form. In certain embodiments, the variant ubiquitin ligase motif comprises changes in at least one lysine residues, two lysine residues, three lysine residues, four lysine residues, five lysine residues, six lysine residues, seven lysine residues, eight lysine residues, nine lysine residues, ten lysine residues, or all lysine residues relative to its “prototype”, “wild-type”, or “naturally-occurring” form. In some embodiments, at least one lysine residues, two lysine residues, three lysine residues, four lysine residues, five lysine residues, six lysine residues, seven lysine residues, eight lysine residues, nine lysine residues, ten lysine residues, or all lysine residues have been modified to alanine. The variant E3 ubiquitin ligase motif may have enhanced, decreased, changed, or substantially similar properties as compared to the naturally occurring E3 ubiquitin ligase motif.

In some embodiments of the compositions and methods according to the present disclosure, the substrate-binding motif and/or the linker has no lysine residues. Thus, in some embodiments, the E3 ubiquitin ligase motif of the degradation domain and the substrate-binding motif of the targeting domain have no lysine residues; the E3 ubiquitin ligase motif of the degradation domain and the linker coupling the degradation domain to the targeting domain have no lysine residues; or the E3 ubiquitin ligase motif of the degradation domain, the substrate-binding motif of the targeting domain, and the linker coupling the degradation domain to the targeting domain have no lysine residues. In certain embodiments, the compositions and methods according to the present disclosure, the degradation domain, the targeting domain, and/or the linker coupling the degradation domain to the targeting domain have no lysine residues. In some embodiments, the chimeric protein molecule comprises no lysine residues.

As described herein, the chimeric protein molecules according to the present disclosure have increased resistance to autoubiquitination compared to a chimeric protein molecule comprising an E3 ubiquitin ligase motif having one or more lysine residues (e.g., a chimeric protein molecule comprising a targeting domain having one or more lysine residues). The term “autoubiquitination” refers to the process by which ubiquitin ligase enzymes catalyze the addition of poly-ubiquitin to themselves.

In some embodiments of the compositions and methods according to the present disclosure, the substrate-binding motif of the targeting domain recognizes a protein substrate, and the E3 ubiquitin ligase motif of the degradation domain permits ubiquination of the protein substrate.

The terms “degradation domain” or “degradation region” are used interchangeably and refer to a portion of a chimeric protein molecule that can facilitate the ubiquitination of a substrate. As described herein, the degradation domain includes an E3 ubiquitin ligase motif without lysine residues.

E3 ubiquitin ligase are a large family of enzymes that catalyze the transfer of ubiquitin to a lysine residue of a substrate protein, forming an isopeptide linkage between the C-terminus of ubiquitin and the substrate protein (see, e.g., Berndsen et al., “New Insights into Ubiquitin E3 Ligase Mechanism,” Nat. Struct. Mol. Biol. 2(4)1:301-307 (2014), which is hereby incorporated by reference in its entirety).

The E3 ubiquitin ligase motif of the degradation domain disclosed herein possesses a functional E3 ligase that is capable of ubiquitinating a substrate without steric disruption from native binding partners. Accordingly, the degradation domain comprising the E3 ubiquitin ligase motif without lysine residues lacks its native substrate recognition region, i.e., the portion of the native E3 ubiquitin ligase that interacts with a natural or native binding partner.

In some embodiments of the compositions and methods according to the present disclosure, the E3 ubiquitin ligase motif of the degradation domain disclosed herein may possess cell-type specific or tissue-specific ligase function. For example, the E3 ubiquitin ligase CHIP is highly expressed in skeletal muscle, heart, pancreas, brain, and placenta, while also detected in kidney, liver, and lung (see, e.g., Ballinger et al. “Identification of CHIP, a Novel Tetratricopeptide Repeat-Containing Protein that Interacts with Heat Shock Proteins and Negatively Regulates Chaperone Functions.” Mol. Cell. Biol. 19:4535-4545 (1999), which is hereby incorporated by reference in its entirety).

In some embodiments of the compositions and methods according to the present disclosure, when the E3 ubiquitin ligase motif of the degradation domain possesses cell-type specific ligase function, the cell type may be skin cells, muscle cells, epithelial cells, endothelial cells, stem cells, umbilical vessel cells, corneal cells, cardiomyocytes, aortic cells, corneal epithelial cells, somatic cells, fibroblasts, keratinocytes, melanocytes, adipose cells, bone cells, osteoblasts, airway cells, microvascular cells, mammary cells, vascular cells, chondrocytes, placental cells, hepatocytes, glial cells, epidermal cells, limbal stem cells, periodontal stem cells, bone marrow stromal cells, hybridoma cells, kidney cells, pancreatic islets, articular chondrocytes, neuroblasts, lymphocytes, or erythrocytes.

The E3 ubiquitin ligase motif of the degradation domain may be a eukaryotic E3 ubiquitin ligase motif, e.g., a U-box motif. U-box motifs are structurally related to the RING finger (see, e.g., Aravind and Koonin, “The U Box is a Modified RING Finger—A Common Domain in Ubiquitination,” Curr. Biol. 10(4):R132-4 (2000) and Ohi et al., “Structural Insights into the U-Box, A Domain Associated with Multi-Ubiquitination,” Nat. Struct. Biol. 10:250-255 (2003), which are hereby incorporated by reference in their entirety). Exemplary U-box ubiquitin ligases include, without limitation, Homo sapiens UBE4A, Homo sapiens UBE4B, Homo sapiens UIP5, Homo sapiens PRP19, Homo sapiens CHIP, and Homo sapiens CYC4; S. cerevisiae Ufd2; and Mus musculus UFD2a, Mus musculus UFD2b, Mus musculus CHIP, Mus musculus KIAA0860) (see, e.g., Hatakeyama et al., “U Box Proteins as a New Family of Ubiquitin-Protein Ligases,” J. Biol. Chem. 276(35): P33111-33120 (2001) and Liu et al., “Act1, A Novel U-Box E3 Ubiquitin Ligase for IL-17R-Mediated Signalling,” Sci. Signal. 2(92): ra63 (2009), which are hereby incorporated by reference in their entirety). In accordance with such embodiments, the U-box motif does not comprise its native (i.e., homologous) substrate-binding motif. For example, in some embodiments of the compositions and methods according to the present disclosure, the eukaryotic E3 ligase motif is a human Carboxyl terminus of Hsc70-Interacting Protein (“CHIP (STUB1)”) whose terminal tetratricopeptide repeat (“TPR”) domain located at the CHIP(STUB1)N-terminus is deleted.

The E3 ubiquitin ligase motif of the degradation domain may be a prokaryotic E3 ligase motif. For example, the E3 ubiquitin ligase motif may be from a bacterial pathogen. In some embodiments, the bacterial pathogen is selected from the group consisting of Shigella, Salmonella, Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia. In some embodiments, the bacterial pathogen is Shigella flexneri.

In some embodiments of the compositions and methods according to the present disclosure, the degradation domain comprises Shigella flexneri E3 ligase, SspH1, SspH2, SlrP, AvrPtoB, LubX, NleG5-1, NleG2-3, LegU1, LegAU13, NIeL, SopA, SidC, XopL, GobX, VirF, GALA, AnkB, or SidE.

In some embodiments of the compositions and methods according to the present disclosure, the degradation domain is a Shigella IpaH protein. Non-limiting examples of Shigella IpaH protein include IpaH9.8, IpaH1.4, IpaH2.5, IpaH4.5, IpaH7.8, IpaH0887, IpaH1389, IpaH2022, IpaH2202, IpaH2610, and IpaH0722.

In some embodiments of the compositions and methods according to the present disclosure, the E3 ubiquitin ligase of the degradation domain comprises or consists of a Shigella IpaH protein whose substrate-binding motif is deleted. For example, the E3 ubiquitin ligase of the degradation domain may comprise Shigella IpaH9.8, Shigella IpaH1.4, Shigella IpaH2.5, Shigella IpaH4.5, Shigella IpaH7.8, Shigella IpaH0887, Shigella IpaH1389, Shigella IpaH2022, Shigella IpaH2202, Shigella IpaH2610, or Shigella IpaH0722 whose native substrate-binding motif is deleted.

The terms “targeting domain” or “target domain” or “targeting moiety” are used interchangeably and refer to a polypeptide region bound covalently or non-covalently to another region within a chimeric protein molecule, which may enhance the concentration of the chimeric protein molecule or composition in a target sub-cellular location, cell, or tissue relative, as compared to the surrounding locations, cells, and/or tissue.

The targeting domains of the present disclosure may be monospecific, bispecific, trispecific, or of greater multispecificity. Multispecific targeting domains can be specific for different epitopes of a substrate or can be specific for both a substrate polypeptide of the present disclosure as well as for heterologous compositions, such as a heterologous polypeptide or solid support material. See, e.g., WO 93/17715; WO 92/08802; WO 91/00360; WO 92/05793; Tutt et al., “Trispecific F(ab′)3 Derivatives that use Cooperative Signaling via the TCR/CD3 Complex and CD2 to Activate and Redirect Resting Cytotoxic T Cells,” J. Immunol. 147:60-69 (1991); U.S. Pat. Nos. 5,573,920, 4,474,893, 5,601,819, 4,714,681, 4,925,648; 6,106,835; Kostelny et al., “Formation of a Bispecific Antibody by the Use of Leucine Zippers,” J. Immunol. 148:1547-1553 (1992), which are hereby incorporated by reference in their entirety. The targeting domains of the present disclosure can be from any animal origin, including birds and mammals. For example, the targeting domains may be from human, marine, rabbit, goat, guinea pig, camel, horse, or chicken.

Techniques for generating targeting domains directed to target substrates are well known to those skilled in the art. Examples of such techniques include, but are not limited to, e.g., those involving display libraries, xeno or humab mice, hybridomas, and the like. Target polypeptides—from which a targeting domain is derived—within the scope of the present disclosure include any polypeptide or polypeptide derivative which is capable of exhibiting antigenicity. Examples include, but are not limited to, substrate and fragments thereof.

In some embodiments of the compositions and methods according to the present disclosure, the targeting domain is derived from or is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

In some embodiments of the compositions and methods according to the present disclosure, the targeting domain is a single chain antibody. Single chain antibodies (“scFv”) are genetically engineered antibodies that consist of the variable domain of a heavy chain at the amino terminus joined to the variable domain of a light chain by a flexible region. In some embodiments, scFv are generated by PCR from hybridoma cell lines that express monoclonal antibodies (mAbs) with known target specificity, or they are selected by phage display from libraries isolated from spleen cells or lymphocytes, and preserve the affinity of the parent antibody. Employing a protocol to identify intracellular substrates, the yeast two-hybrid technology serves to identify candidate scFv—protein interactions. Such a system is useful to predict whether or not a scFv will be able to recognize its target substrate in vivo (see Portner-Taliana et al., “Identification of Protein Single chain Antibody Interactions In Vivo Using Two-hybrid Protocols,” Protein-Protein Interactions: A Molecular Cloning Manual, Cold Spring Harbor Laboratory Press, Chapter 24 (2002), which is hereby incorporated by reference in its entirety.

Typically, scFv, hybrid antibodies or hybrid antibody fragments that are cloned into a display vector can be selected against the appropriate antigen to identify variants that maintained good binding activity, because the antibody or antibody fragment will be present on the surface of the phage or phagemid particle (see, e.g., Barbas III et al., Phage Display, A Laboratory Manual (Cold Spring Harbor Laboratory Press, Cold Spring Harbor, N.Y., 2001), which is hereby incorporated by reference in its entirety). However, other vector formats could be used for this process, such as cloning the antibody fragment library into a lytic phage vector (modified T7 or Lambda Zap systems) for selection and/or screening.

In some embodiments of the compositions and methods according to the present disclosure, where the targeting domain is a monobody, the monobody may be a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), E13.4.3 (EGFR), EI4.2.1 (EGFR), EI4.4.2 (EGFR), E16.2.6 (EGFR), E16.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

The targeting domain according to the present disclosure comprises a substrate-binding motif which is heterologous to the E3 ubiquitin ligase of the degradation domain. The substrate-binding motif may recognize a protein substrate (e.g., a biomolecule). In accordance with such embodiments the E3 ubiquitin ligase motif of the degradation domain permits ubiquination of the protein substrate (e.g., the biomolecule).

As described herein, a known or unknown substrate (e.g., a protein substrate) may be bound by the substrate-binding motif of the targeting domain for subsequent ubiquitination via the E3 ubiquitin ligase motif of the degradation domain. The substrates include, but are not limited to, intracellular substrates, extracellular substrates, modified substrates, glycosylated substrates, farnesylated substrates, post translationally modified substrates, phosphorylated substrates, and other modifications known in the art. In some embodiments, the substrate-binding motif binds an intracellular substrate, e.g., an intracellular protein substrate.

In some embodiments of the compositions and methods according to the present disclosure, the substrate-binding motif binds a substrate selected from the group consisting of, but not limited to, β-galactosidase, fluorescent protein, histone protein, nuclear localization signal (NLS), H-Ras protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, or pathogenic proteins.

In some embodiments of the compositions and methods according to the present disclosure, the substrate-binding motif binds a fluorescent protein selected from the group consisting of green fluorescent protein, emerald fluorescent protein, venus fluorescent protein, cerulean fluorescent protein, and enhanced cyan fluorescent protein.

Although targeting domains possess intrinsic binding interactions, e.g., secondary, tertiary, or quaternary flexibility, there must still be flexibility with respect to the association with the degradation domain. In this regard, absence adequate spacing, it is possible for the E3 ubiquitin ligase motif of the degradation domain to sterically hinder the substrate-targeting domain interaction. As such, the present disclosure employs polypeptide linkers of sufficient length to prevent the steric disruption of binding between the targeting domain and the substrate, in some embodiments.

In some embodiments of the compositions and methods according to the present disclosure, the targeting domain is covalently attached to the degradation domain via a linker that may be cleavable or non-cleavable under physiological conditions. The linker can entail an organic moiety comprising a nucleophilic or electrophilic reacting group which allows covalent attachment of the degradation domain to the targeting domain. In some embodiments, the linker is an enol ether, ketal, imine, oxime, hydrazone, semicarbazone, acylimide, or methylene radical. The linker may be an acid-cleavable linker, a hydrolytically cleavable linker, or enzymatically-cleavable linker, in some embodiments. Accordingly, In some embodiments of the compositions and methods according to the present disclosure, when the linker is cleavable, the linker may be enzymatically or hydrolytically cleavable.

Peptide-based linking groups are cleaved by enzymes such as peptidases and proteases in cells. Peptide-based cleavable linking groups are peptide bonds formed between amino acids to yield oligopeptides, e.g., dipeptides, tripeptides, and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide-based cleavage group is generally limited to the peptide bond, i.e., the amide bond, formed between amino acids yielding peptides and proteins and does not include the entire amide functional group. Peptide cleavable linking groups have the general formula —NHCHR1C(O)NHCHR2C(O)—, where R1 and R2 are the R groups of the two adjacent amino acids. These candidates can be evaluated using methods analogous to those described above.

For in vitro applications, appropriate linkers, which can be cross-linking agents for use for conjugating a polypeptide to a solid support, include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. See Brown et al., Mol. Divers 4-12 (1995); Rothschild et al., Nucl. Acids Res. 24:351-66 (1996); and U.S. Pat. No. 5,643,722), which are hereby incorporated by reference in their entirety.

In some embodiments of the compositions and methods according to the present disclosure, the linker is not cleavable.

In some embodiments of the compositions and methods according to the present disclosure, the linker is heterologous to the degradation domain and the targeting domain.

An antibody, polypeptide, or fragment thereof, such as a targeting domain, can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoracetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

It will be readily apparent to the skilled artisan that the methods and techniques described above can be employed for the chimeric protein molecule of the present disclosure, including its constituent parts, e.g., degradation domains, E3 ubiquitin ligase motif regions, targeting domains, and substrate-binding motifs, and modifications thereof, as well as the linker molecules, as described above.

A further aspect of the present disclosure relates to a composition comprising the chimeric protein molecule and a pharmaceutically-acceptable carrier. Such compositions generally entail recombinant or substantially purified chimeric protein molecules and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the protein compositions (see, e.g., Remington's Pharmaceutical Sciences, Mack Publishing Co., Easton, Pa. 18th ed. (1990), which is hereby incorporated by reference in its entirety). The pharmaceutical compositions are generally formulated as sterile, substantially isotonic and in full compliance with all Good Manufacturing Practice (GMP) regulations of the U.S. Food and Drug Administration.

The compositions of the present disclosure can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The compositions of the present disclosure may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compositions may be incorporated with excipients and used in the form of tablets, capsules, elixirs, suspensions, syrups, and the like. Such compositions and preparations should contain at least 0.1% of active compound. The percentage of the composition in these compositions may, of course, be varied and may conveniently be between about 2% to about 60% of the weight of the unit. Preferred compositions according to the present disclosure are prepared so that an oral dosage unit contains between about 1 and 250 mg of the chimeric protein molecule according to the present disclosure.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

These compositions may also be administered parenterally. Solutions or suspensions of the present compositions can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The composition of the present disclosure may also be administered directly to the airways in the form of an aerosol. For use as aerosols, the compositions of the present disclosure in solution or suspension may be packaged in a pressurized aerosol container together with suitable propellants, for example, hydrocarbon propellants like propane, butane, or isobutane with conventional adjuvants. The materials of the present disclosure also may be administered in a non-pressurized form such as in a nebulizer or atomizer.

The compositions of the present disclosure may further contain, in some embodiments, a second agent or pharmaceutical composition selected from the non-limiting group of anti-inflammatory agents, antidiabetic agents, hypolipidemic agents, chemotherapeutic agents, antiviral agents, antibiotics, metabolic agents, small molecule inhibitors, protein kinase inhibitors, adjuvants, apoptotic agents, proliferative agents, organotropic targeting agents, immunological agents, antigens from pathogens, such as viruses, bacteria, fungi and parasites, optionally in the form of whole inactivated organisms, peptides, proteins, glycoproteins, carbohydrates, or combinations thereof, any examples of pharmacological or immunological agents that fall within the above-mentioned categories and that have been approved for human use that may be found in the published literature, any other bioactive component, or any combination of any of these.

In some embodiments, the composition further contains a second agent selected from the group consisting of an anti-inflammatory agent, an antidiabetic agent, a hypolipidemic agent, a chemotherapeutic agent, an antiviral agent, an antibiotic, a metabolic agent, a small molecule inhibitor, a protein kinase inhibitor, adjuvants, apoptotic agents, a proliferative agent, and organotropic targeting agents, and any combination thereof.

The importance of E3 ligases, and functional domains thereof (e.g., E3 ubiquitin ligase motifs), is highlighted by the number of normal cellular processes they regulate, and underlies the attendant diseases associated with loss of function or inappropriate targeting (see, e.g., Ardley et al., “E3 Ubiquitin Ligases,” Essays Biochem. 41:15-30 (2005), which is hereby incorporated by reference in its entirety). For example, recessive mutations in the parkin gene are a well-known cause of familial Parkinson disease, and therefore it is of significant interest that CHIP also interacts with the products of several other familial Parkinson disease genes, such as SNCA (which encodes α-synuclein; and LRRK2. Id. The involvement of CHIP in interacting and/or ubiquitinating other proteins also implicates nervous system and neurodegenerative diseases, such as Tau and APP (Alzheimer disease), Malin (Lafora disease) and ataxin-1 and ataxin-3 (associated respectively to spinocerebellar ataxia types 1 and 3). See id. Consistent with a highly pleiotropic phenotype, CHIP null mutant mice show shortened life span, accelerated aging and anomalous oxidative stress and protein quality levels (see, e.g., Marin, I., “Ancient Origin of Animal U-box Ubiquitin Ligases,” BMC Evolutionary Biology 10:331, pp. 1-15 (2010), which is hereby incorporated by reference in its entirety).

Another aspect of the present disclosure relates to a method of treating a disease. This method involves administering a composition according to the present disclosure to a subject having a disease, where the subject to whom the composition is administered has an increased expression level of a substrate (e.g., a biomolecue) compared to a subject not afflicted with the disease.

The treatment methods of the present disclosure may involve administering a composition according to the present disclosure to a subject, where the disease possesses a measurable phenotype. In some embodiments, the phenotype of the disease involves an increased expression level of a substrate compared to the phenotype from a subject not afflicted with the disease. In this respect, chimeric protein molecules contained in the pharmaceutical compositions of the present disclosure are efficacious against treating or alleviating the symptoms from a disease characterized by a phenotypic increase in the expression level of one or more substrates compared to the phenotype from a subject not afflicted with the disease.

Non-limiting examples of diseases that can be treated or prevented in the context of the present disclosure, include, cancer, metastatic cancer, solid cancers, invasive cancers, disseminated cancers, breast cancer, lung cancer, NSCLC cancer, liver cancer, prostate cancer, brain cancer, pancreatic cancer, lymphatic cancer, ovarian cancer, endometrial cancer, cervical cancer, and other solid cancers known in the art, blood cell malignancies, lymphomas, leukemias, myelomas, stroke, ischemia, myocardial infarction, congestive heart failure, stroke, ischemia, peripheral vascular disease, alcoholic liver disease, cirrhosis, Parkinson's disease, Alzheimer's disease, diabetes, cancer, arthritis, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, septic shock, multiple organ dysfunction syndrome, rheumatoid arthritis, trauma, stroke, heart infarction, systemic autoimmune disease, chronic hepatitis, overweight, and/or obesity, or any combination thereof.

In some embodiments, the disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

When used in vivo for therapy, the compositions are administered to the subject in effective amounts, i.e., amounts that have desired therapeutic effect. The dose and dosage regimen will depend upon the degree of the disease in the subject, the characteristics of the particular chimeric protein molecule used, e.g., its therapeutic index, the subject, and the subject's history. The effective amount may be determined during pre-clinical trials and clinical trials by methods familiar to physicians and clinicians. An effective amount of a peptide useful in the methods may be administered to a mammal in need thereof by any of a number of well-known methods for administering pharmaceutical compounds.

Dosage, toxicity and therapeutic efficacy of the compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices may be desirable. While compositions that exhibit toxic side effects may be used, care should be taken to design a delivery system that targets such compositions to the site of affected tissue in order to minimize potential damage to uninfected cells and, thereby, reduce side effects.

In some embodiments, administering the compositions of the present disclosure is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

In some embodiments, suitable in vitro or in vivo assays are performed to determine the effect of the chimeric protein molecules and compositions of the present disclosure and whether administration is indicated for treatment. Compositions for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Any method known to those in the art for contacting a cell, organ or tissue with a composition may be employed. In vivo methods typically include the administration of a chimeric protein molecule or composition, such as those described above, to a mammal, suitably a human. When used in vivo for therapy, the chimeric protein molecules or compositions are administered to the subject in effective amounts, as described herein. Results can be ascertained as per the empirical variables set forth at the outset of the methods described herein.

In vitro methods typically include the assaying the effect of chimeric protein molecule or composition, such as those described above, on a sample or extract. In some embodiments, chimeric protein molecule efficacy can be determined by assessing the effect on substrate degradation, i.e., the ability of the chimeric protein molecules and compositions to exert a phenotypic change in a sample. Such methods include, but are not limited to, immunohistochemistry, immunofluorescence, ELISPOT, ELISA, or RIA. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods.

Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, immunohistochemistry, fluorescence microscopy, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomolecule) with an antibody to the molecule of interest or contacting an antibody to a molecule of interest (such as antibodies to the disclosed biomolecule) with a molecule that can be bound by the antibody, as the case may be, under conditions effective to allow the formation of immunocomplexes. In this regard, the skilled artisan will be able to assess the presence and or level of specific biomolecules in a given sample. Subsequently, the chimeric protein molecule compositions of the present disclosure are added to the assay. Thereafter, the level of biomolecule can be assessed, i.e., the presence or level thereof, using the immunoassays described herein to determine the post-treatment phenotypic effect.

Immunoassays can include methods for detecting or quantifying the amount of a biomolecule of interest in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label (see, e.g., U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

Another aspect of the present disclosure relates to a method for protein substrate silencing. This method involves selecting a protein substrate to be silenced and providing a chimeric protein molecule according to the present disclosure. This method further involves contacting the protein substrate and the chimeric protein molecule under conditions effective to permit the formation of a protein substrate-molecule complex, where the complex mediates degradation of the protein substrate to be silenced. In some embodiments, the complex mediates the degradation by post-translational ubiquitination of the substrate.

The methods involve silencing one or more substrates as previously described herein. In some embodiments, the protein substrate is selected from the group consisting of β-galactosidase, fluorescent protein, histone protein, nuclear localization signal (NLS), H-Ras protein, SHP2 protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

In some embodiments, the substrate is a fluorescent protein selected from the group consisting of green fluorescent protein, emerald fluorescent protein, venus fluorescent protein, cerulean fluorescent protein, and enhanced cyan fluorescent protein.

Another aspect of the present disclosure relates to forming a ribonucleoprotein. This method involves providing a mRNA encoding the chimeric protein molecule according to the present disclosure and providing one or more polyadenosine binding proteins (“PABP”). This method further involves assembling a ribonucleoprotein complex from the mRNA and the one or more PABPs. In some embodiments, the mRNA comprises a 3′-terminal polyadenosine (poly A) tail.

Another method of the present disclosure relates to a method of screening agents for therapeutic efficacy against a disease. This method involves providing a biomolecule whose presence is mediated by a disease state. A test agent comprising (i) a degradation domain including an E3 ubiquitin ligase motif without lysine residues, (ii) a targeting domain comprising a substrate-binding motif which is heterologous to the E3 ubiquitin ligase motif, and (iii) a linker coupling the degradation domain to the targeting domain are provided. The biomolecule and the test agent are contacted under conditions effective for the test agent to facilitate degradation of the biomolecule. The level of the biomolecule, as a result of the contacting, is determined and the test agent which, based on the determining, decreases the level of the biomolecule is identified as being a candidate for therapeutic efficacy against the disease. Accordingly, in some embodiments, the identified test agent comprises a substrate-binding motif which binds the biomolecule.

There are a myriad of diseases in which the degree of overabundance of certain biomolecules are known to be indicative of whether a subject is afflicted with a disease or is likely to develop a disease (see, e.g., Anderson et al., “Discovering Robust Protein Biomarkers for Disease from Relative Expression Reversals in 2-D DIGE Data,” Proteomics 7:1197-1207 (2007), which is hereby incorporated by reference in its entirety). Accordingly, in some embodiments, the biomolecule is associated with cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, or obesity, and any combination thereof.

In some embodiments, the test agent is a chimeric protein molecule according to the present disclosure.

As described herein, the test agent comprises a degradation domain coupled to a targeting domain by a linker. In some embodiments, the linker is a polypeptide linker of sufficient length to prevent the steric disruption of binding between said targeting domain and said biomolecule. The linker may be heterologous to the degradation domain and the targeting domain.

In some embodiments, the substrate-binding motif and/or the linker has no lysine residues. For example, the E3 ubiquitin ligase motif of the degradation domain and the substrate-binding motif of the targeting domain may have no lysine residues; the E3 ubiquitin ligase motif of the degradation domain and the linker coupling the degradation domain to the targeting domain may have no lysine residues; or the E3 ubiquitin ligase motif of the degradation domain, the substrate-binding motif of the targeting domain, and the linker coupling the degradation domain to the targeting domain may have no lysine residues.

In some embodiments, the degradation domain, the targeting domain, and/or the linker coupling the degradation domain to the targeting domain have no lysine residues. In some embodiments, the test agent comprises no lysine residues.

As described herein, the test agent may have increased resistance to autoubiquitination compared to a test agent comprising an E3 ubiquitin ligase motif having one or more lysine residues.

Identifying the test agent may be carried out with respect to a standard biomolecule level in a subject not afflicted with said disease.

In some embodiments, identifying the test agent may be carried out with a plurality of test agents.

In accordance with this aspect of the disclosure, the E3 ubiquitin ligase motif may be a eukaryotic E3 ligase motif (e.g., a U-box motif). Exemplary eukaryotic E3 ligase motifs are described in detail supra. In some embodiments, the eukaryotic E3 ligase motif is human Carboxyl terminus of Hsc70-Interacting Protein (“CHIP (STUB1)”) whose TPR domain located at the CHIP(STUB1)N-terminus is deleted.

In accordance with this aspect of the disclosure, the E3 ubiquitin ligase motif may be a prokaryotic E3 ligase motif. For example, the prokaryotic E3 ligase motif may be from a bacterial pathogen. In some embodiments, when the prokaryotic E3 ligase is from a bacterial pathogen selected from the group consisting of Shigella, Salmonella, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia. In some embodiments, the bacterial pathogen is Shigella flexneri.

In some embodiments, the degradation domain is from a bacterial pathogen and comprises Shigella flexneri E3 ligase, SspH1, SspH2, SlrP, AvrPtoB, LubX, N1eG5-1, N1eG2-3, LegU1, LegAU13, NIeL, SopA, SidC, XopL, GobX, VirF, GALA, AnkB, or SidE. In certain embodiments, the degradation domain is a Shigella IpaH protein. Suitable Shigella IpaH protein may be selected from the group consisting of IpaH9.8, IpaH1.4, IpaH2.5, IpaH4.5, IpaH7.8, IpaH0887, IpaH1389, IpaH2022, IpaH2202, IpaH2610, and IpaH0722.

Targeting domains and substrate-binding motifs for use in methods of the present disclosure are described in detail supra. In some embodiments, the targeting domain and/or the substrate-binding motif is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d. Accordingly, the targeting domain and/or the substrate-binding motif may be a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), E13.4.3 (EGFR), EI4.2.1 (EGFR), EI4.4.2 (EGFR), E16.2.6 (EGFR), E16.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP- 76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

In accordance with this aspect of the disclosure, the substrate-binding motif may bind a substrate selected from the group consisting of β-galactosidase, fluorescent protein, histone protein, nuclear localization signal (NLS), H-Ras protein, SHP2 protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

In some embodiments, the substrate-binding motif binds a fluorescent protein selected from the group consisting of green fluorescent protein, emerald fluorescent protein, venus fluorescent protein, cerulean fluorescent protein, and enhanced cyan fluorescent protein.

In some embodiments, the substrate-binding motif binds a fusion protein comprising a fluorescent protein. Suitable fluorescent proteins are identified supra.

Another aspect of the present disclosure relates to a method of screening for disease biomarkers. This method involves providing a sample of diseased cells expressing one or more ligands. A plurality of chimeric protein molecules comprising (i) a degradation domain including an E3 ubiquitin ligase motif without lysine residues, (ii) a targeting domain comprising a substrate-binding motif which is heterologous to the E3 ubiquitin ligase motif, and (iii) a linker coupling the degradation domain to the targeting domain are provided. This method further involves contacting the sample with the plurality of chimeric protein molecules under conditions effective for the diseased cells to fail to proliferate in the absence of the chimeric protein molecule, determining which of the chimeric protein molecules permit the diseased cells to proliferate, and identifying, as biomarkers for the disease, based on the determining the ligands which bind to the chimeric protein molecules and permit diseased cells to proliferate.

Suitable chimeric protein molecules, degradation domains, E3 ubiquitin ligase moieties, targeting domains, and substrate-binding motifs, and likers are described in detail supra. In some embodiments, the linker is heterologous to the degradation domain and the targeting domain.

In some embodiments, the plurality of chimeric protein molecules and/or the linker has no lysine residues. In certain embodiments, the plurality of chimeric protein molecules has increased resistance to autoubiquitination compared to a plurality of chimeric protein molecules comprising an E3 ubiquitin ligase motif having one or more lysine residues.

Many, if not all diseases, are complex and multifactorial. When considering neurodegeneration, for example, substantial neuronal cell loss occurs before pathologic presentation. Screening for and developing such drugs—to treat neurodegenerative diseases—is further stymied by ancillary therapies which ameliorate the symptoms. Thus, target detection is obfuscated by prior therapeutic administration, which, may in turn, slow disease progression and further confound treatment regimes. In this way, the present disclosure provides new, inventive, screening methods for elucidation of disease biomarkers by employing phenotypic screening analyses (see, e.g., Pruss, R. M., “Phenotypic Screening Strategies for Neurodegenerative Diseases: A Pathway to Discover Novel Drug Candidates and Potential Disease Targets or Mechanisms,” CNS & Neurological Disorders—Drug Targets, 9, 693-700 (2010), which is hereby incorporated by reference in its entirety).

Phenotypic screening involves using an appropriate sample, e.g., class of cells, cell extract, neurons, tissue, and the like, from a patient afflicted with a disease and subjecting the sample to one or more chimeric protein molecules as described herein. Subsequently, the sample is screened for viability, proliferation, cell processes and/or phenotypic characteristic of the diseased cell, e.g., shrinking, loss of membrane potential, morphological changes, and the like. See id. Image analysis software allows for cell bodies or other objects to empirically assess the results. Hits coming from the screen may maintain cell survival by stimulating survival pathways, mimicking trophic factors, or inhibiting death signaling. Higher content screening and profiling in target-directed secondary assays can then be used to identify targets and mechanisms of action of promising hits.

Examples of diseases conditions from which a biomarker screening analysis can be performed include the diseases described above. In some embodiments, the disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

In some embodiments, the method of screening for disease biomarkers includes a plurality of chimeric protein molecules, where the molecules possess an E3 ubiquitin ligase motif, as described supra. In some embodiments, the biomarker screening method includes a plurality of chimeric protein molecules possessing a targeting domain, as described above. The screening methods of the present disclosure employ polypeptide linkers of sufficient length to prevent the steric disruption of binding between the targeting domain and the ligand.

Once a chimeric protein molecule is determined to provide a therapeutic indication, the biomarker is isolated using the targeting domain region (or the entire chimeric protein molecule) to immunoprecipitate the biomarker, from a sample, which is subsequently identified using methods well known in the art. Biomarker isolation and purification methods include, but are not limited to, for example, HPLC or FPLC chromatography using size-exclusion or affinity-based column resins (see, e.g., Sambrook, et al. 1989, Cold Spring Harbor Laboratory Press, which is hereby incorporated by reference in its entirety).

Active fragments, derivatives, or variants of the polypeptides of the present disclosure may be recognized by, for example, the deletion or addition of amino acids that have minimal influence on the properties, secondary structure, and biological activity of the polypeptide. For example, a polypeptide may be joined to a signal (or leader) sequence at the N-terminal end of the protein which co-translationally or post-translationally directs sub-cellular or extracellular localization of the protein.

The biomarker can then be elucidated using techniques known in the art. In some embodiments, determining the identity of the biomarker is performed using MALDI-TOF, mass spectrometry, mass spectroscopy, protein sequencing, antibody interactions, western blot, immunoassay, ELISA, chromatographic techniques, reverse proteomics, immunoprecipitations, radioimmunoassay, and immunofluorescence, or any combinations thereof.

Suitable mass spectrometric techniques for the study and identification of proteins include, laser desorption ionization mass spectrometry and electrospray ionization mass spectrometry. Within the category of laser desorption ionization (LDI) mass spectrometry (MS), both matrix assisted LDI (MALDI) and surface assisted LDI (SELDI) time-of-flight (TOF) MS may be employed. SELDI TOF-MS is particularly well-suited for use in the present methods because it provides attomole sensitivity for analysis, quantification of low abundant proteins (pg-ng/ml) and highly reproducible results.

The methods described herein can be performed, e.g., by utilizing pre-packaged kits comprising at least one reagent, e.g., a chimeric protein molecule or composition described herein, which can be conveniently used, e.g., in clinical settings to treat subjects exhibiting symptoms of a disease or illness involving an overexpressed substrate, biomolecule, or biomarker.

Another aspect of the present disclosure relates to a mRNA molecule encoding a chimeric protein molecule according to the present disclosure. Suitable chimeric protein molecules are described in detail supra. The mRNA molecule may comprise a 3′-terminal polyadenosine (poly A) tail.

A further aspect of the present disclosure relates to a vector encoding a mRNA molecule according to the present disclosure.

The vector may be an expression vector. In general, expression vectors useful in recombinant DNA techniques are often in the form of plasmids. However, the present disclosure is intended to include such other forms of expression vectors that are not technically plasmids, such as viral vectors, e.g., replication defective retroviruses, adenoviruses and adeno-associated viruses, which serve equivalent functions. Such viral vectors permit infection of a host cell and expression in that host cell of, e.g., a mRNA and its subsequent translation to a protein.

In some embodiments, the vector is a viral vector, e.g., an adenoviral vector, an adeno-associated virus vector, or a lentiviral vector.

The vectors may comprise eukaryotic promoter systems capable of transforming or transfecting eukaryotic host cells. Once the vector has been incorporated into the appropriate host, the host is maintained under conditions suitable for high level expression of the nucleotide sequences encoding the chimeric protein molecule according to the present disclosure. Vectors can also encode a signal peptide, e.g., pectate lyase, useful to direct the secretion of extracellular antibody fragments (see U.S. Pat. No. 5,576,195, which is hereby incorporated by reference in its entirety).

Expression of the chimeric protein molecules of the present disclosure in prokaryotes is most often carried out in E coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression; (ii) to increase the solubility; and (iii) to aid in purification by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their endogenous recognition sequences, include Factor Xa, thrombin and enterokinase. Typical fusion expression vectors include pGEX (Pharmacia Biotech Inc; Smith and Johnson, Gene 67:31-40 (1988), which is hereby incorporated by reference in its entirety), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E coli expression vectors include pTrc (Amrann et al., “Tightly Regulated tac Promoter Vectors useful for the Expression of Unfused and Fused Proteins in Escherichia coli,” Gene 69(2):301-315 (1988), which is hereby incorporated by reference in its entirety) and pET 11d (Studier et al., Gene Expression Technology: Methods In Enzymology 185, Academic Press, San Diego, Calif. 60-89 (1990), which is hereby incorporated by reference in its entirety). Methods for targeted assembly of distinct active peptide or protein domains to yield multifunctional polypeptides via polypeptide fusion has been described by U.S. Pat. Nos. 6,294,353 and 6,692,935, which are hereby incorporated by reference in their entirety. One strategy to maximize recombinant polypeptide expression, e.g., a chimeric protein molecule of the present disclosure, in E. coli is to express the polypeptide in host bacteria with an impaired capacity to proteolytically cleave the recombinant chimera (see, e.g., Gottesman, Gene Expression Technology: Methods In Enzymology 185, Academic Press, San Diego, Calif. 119-128 (1990), which is hereby incorporated by reference in its entirety).

Expression of the chimeric protein molecules of the present disclosure may be carried out in mammalian cells using a mammalian expression vector. Examples of mammalian expression vectors include, e.g., but are not limited to, pCDM8 (Seed, “An LFA-3 cDNA Encodes a Phospholipid-Linked Membrane Protein Homologous to its Receptor CD2,” Nature 329:840 (1987), which is hereby incorporated by reference in its entirety) and pMT2PC (Kaufman, et al., “Translational Efficiency of Polycistronic mRNAs and their Utilization to Express Heterologous Genes in Mammalian Cells,” EMBO J. 6:187-195 (1987), which is hereby incorporated by reference in its entirety). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells useful for expression of the chimeric protein molecules according to the present disclosure (see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., (1989), which are hereby incorporated by reference in their entirety).

A further aspect of the present disclosure relates to an encapsulated nucleic acid molecule comprising: (i) a mRNA molecule according to the present disclosure or a vector according to the present disclosure and (ii) a protein and/or polymer complex.

In some embodiments, the nucleic acid is a mRNA molecule, wherein said mRNA molecule comprises a 3′-terminal polyadenosine (poly A) tail, and wherein said protein complex comprises one or more polyadenosine binding proteins (“PABP”).

In some embodiments, the protein and/or polymer complex comprises one or more antibodies, antibody derivatives, antibody-drug conjugates, phage proteins, ubiquibodies, or combinations thereof.

In some embodiments, the protein and/or polymer complex forms a nanoplex.

### EXAMPLES

The examples below are intended to exemplify the practice of embodiments of the disclosure but are by no means intended to limit the scope thereof.

### Materials and Methods

### Plasmid Construction

Three plasmids were constructed for evaluating long-lived degradation of EGFP. One plasmid, pcDNA3-GS2-IpaH9.8ΔLRR, encoded the EGFP-specific FN3 monobody GS2 fused to the N-terminus of the Shigella flexneri E3 ubiquitin ligase IpaH9.8 lacking its native substrate-binding domain. The resulting construct was an EGFP-specific ubiquibody, hereafter GS2-uAb. The second plasmid, pcDNA3-GS2-IpaH9.8ΔLRR(K-free), was identical to the first except that all of the lysine residues in the GS2 and IpaH9.8ΔLRR domains were mutated to alanine residues. Note, there are 2 lysine residues in GS2 and 9 lysine residues in IpaH9.8ΔLRR. The resulting construct was a lysine-free, EGFP-specific ubiquibody, hereafter GS2-uAb(K-free). The third plasmid, pcDNA3-GS2-IpaH9.8ΔLRR(K-free/C337A), was identical to the second except that it carried an active-site mutation, C337A, that inactivated ubiquitination activity. The resulting construct was a lysine-free, EGFP-specific ubiquibody, hereafter GS2-uAb(K-free/C337A) that was catalytically inactive and thus unable to degrade EGFP. A fourth plasmid, pcDNA3-EGFP which encoded the target EGFP protein, was also constructed and served as a reporter for ubiquibody degradation activity in HEK293-T cells. The GS2-IpaH9.8ΔLRR and GS2-IpaH9.8ΔLRR(C337A) constructs were generated previously (Ludwicki et al., “Broad-Spectrum Proteome Editing with an Engineered Bacterial Ubiquitin Ligase Mimic,” ACS Central Science 5(5):852-866 (2019), which is hereby incorporated by reference in its entirety). The lysine-free version of GS2-IpaH9.8ΔLRR was designed such that all 12 lysines in the original GS2-IpaH9.8ΔLRR were replaced with alanine (specifically, at locations: K8A, K55A, K106A, K155A, K190A, K229A, K252A, K260A, K305A, K319A, K336A). The designed gene sequence with a 6×-His tag added at the C-terminus was ordered as a gblock (Integrated DNA Technologies, IDT) and cloned into the pcDNA3 vector via Gibson assembly. PCR was performed to obtain backbone and insert fragments with 30-bp overlap both upstream and downstream of the intended insertion location. After gel purification, insert and backbone with a ratio of 2:1 was combined with Gibson NEB Gibson Assembly Master Mix at 50° C. for 30 min. 2 μL of the mixture was used to transform electrocompetent E. coli DH5a cells. The catalytically inactivated mutant GS2-IpaH9.8ΔLRR(K-free/C337A) was generated by performing site-directed mutagenesis on the cysteine at amino acid position 337 to alanine on the cloned lysine-free GS2-IpaH9.8ΔLRR(K-free) construct.

Two ubiquibody plasmids were constructed for evaluating long-lived degradation of human FOXP3. One plasmid, pcDNA3-D16-CHIPΔTPR, encoded the FOXP3-specific peptide D16 fused to the N-terminus of the human E3 ubiquitin ligase CHIP lacking its native substrate-binding domain. The resulting construct was a FOXP3-specific ubiquibody, hereafter D16-uAb. The second plasmid, pcDNA3-D16-CHIPΔTPR(K-free), was identical to the first except that all of the lysine residues in the CHIPΔTPR domain were mutated to arginine residues. Note, there are no lysines in the D16 peptide sequence and 11 lysines in CHIPΔTPR. The resulting construct was a lysine-free, FOXP3-specific ubiquibody, hereafter D16-uAb(K-free). A third plasmid, pcDNA3-FOXP3-GFP which encoded the target protein fused to superfolder GFP, was also constructed and served as a reporter for ubiquibody degradation activity in HEK293-T cells. The designed gene sequences for the lysine-free CHIPΔTPR constructs were ordered as a gblock (Integrated DNA Technologies, IDT). PCR was run using Vent polymerase together with the gBlock sequences as template DNA and gene specific primers. PCR products were digested and then ligated into the pcDNA3 plasmid. Ligated plasmids were used to transform electrocompetent E coli DH5a cells. Isolated plasmids from randomly chosen individual colonies were confirmed by sequencing at the Genomics Core Facility of the Cornell Biotechnology Resource Center (BRC). The sequences for each of the ubiquibody constructs are shown in Table 1.

TABLE 1
Ubiquibody Constructs

| | | SEQ ID |
|---|---|---|
| Ubiquibody | Sequence | NO. |
| GS2- | ATGGTTTCTTCTGTTCCGACCAAACTGGAAGTTGTTGC | 1 |
| IpaH9.8ΔLRR | TGCGACCCCGACTAGCCTGCTGATCAGCTGGGATGCT | |
| | CCTGCAGTTACCGTCGATCATTACTATATCACGTACGG | |
| | TGAAACCGGTCATTACTGGTACTACCAGGCATTCGCG | |
| | GTACCTGGTTCCAAGTCTACTGCTACCATCAGCGGCCT | |
| | GAGCCCGGGTGTCGACTATACCATCACTGTATACGCA | |
| | CCGTTCTCTGTTCCGGTTATGAGCCCAATCTCGATTAA | |
| | CTACCGTACCGCTAGCCTGGCTGATGCCGTGACAGCA | |
| | TGGTTCCCGGAAAACAAACAATCTGATGTATCACAGA | |
| | TATGGCATGCTTTTGAACATGAAGAGCATGCCAACAC | |
| | CTTTTCCGCGTTCCTTGACCGCCTTTCCGATACCGTCT | |
| | CTGCACGCAATACCTCCGGATTCCGTGAACAGGTCGC | |
| | TGCATGGCTGGAAAAACTCAGTGCCTCTGCGGAGCTT | |
| | CGACAGCAGTCTTTCGCTGTTGCTGCTGATGCCACTGA | |
| | GAGCTGTGAGGACCGTGTCGCGCTCACATGGAACAAT | |
| | CTCCGGAAAACCCTCCTGGTCCATCAGGCATCAGAAG | |
| | GCCTTTTCGATAATGATACCGGCGCTCTGCTCTCCCTG | |
| | GGCAGGGAAATGTTCCGCCTCGAAATTCTGGAGGATA | |
| | TTGCCCGGGATAAAGTCAGAACTCTCCATTTTGTGGAT | |
| | GAGATAGAAGTCTACCTGGCCTTCCAGACCATGCTCG | |
| | CAGAGAAACTTCAGCTCTCCACTGCCGTGAAGGAAAT | |
| | GCGTTTCTATGGCGTGTCGGGAGTGACAGCAAATGAC | |
| | CTCCGCACTGCCGAAGCCATGGTCAGAAGCCGTGAAG | |
| | AGAATGAATTTACGGACTGGTTCTCCCTCTGGGGACC | |
| | ATGGCATGCTGTACTGAAGCGTACGGAAGCTGACCGC | |
| | TGGGCGCAGGCAGAAGAGCAGAAATATGAGATGCTG | |
| | GAGAATGAGTACCCTCAGAGGGTGGCTGACCGGCTGA | |
| | AAGCATCAGGTCTGAGCGGTGATGCGGATGCGGAGA | |
| | GGGAAGCCGGTGCACAGGTGATGCGTGAGACTGAAC | |
| | AGCAGATTTACCGTCAGCTGACTGACGAGGTACTGGC | |
| | CCTGCGATTGTCTGAAAACGGCTCACAACTGCACCAT | |
| | TCACCTGCAGGAGCAGACTACAAGGACGATGACGAC | |
| | AAGGGACATCATCATCATCATCACTAA | |
| GS2- | ATGGTTTCTTCTGTTCCGACCGCACTGGAAGTTGTTGC | 2 |
| IpaH9.8ΔLRR | TGCGACCCCGACTAGCCTGCTGATCAGCTGGGATGCT | |
| (K-free) | CCTGCAGTTACCGTCGATCATTACTATATCACGTACGG | |
| | TGAAACCGGTCATTACTGGTACTACCAGGCATTCGCG | |
| | GTACCTGGTTCCGCGTCTACTGCTACCATCAGCGGCCT | |
| | GAGCCCGGGTGTCGACTATACCATCACTGTATACGCA | |
| | CCGTTCTCTGTTCCGGTTATGAGCCCAATCTCGATTAA | |
| | CTACCGTACCGCTAGCCTGGCTGATGCCGTGACAGCA | |
| | TGGTTCCCGGAAAACGCACAATCTGATGTATCACAGA | |
| | TATGGCATGCTTTTGAACATGAAGAGCATGCCAACAC | |
| | CTTTTCCGCGTTCCTTGACCGCCTTTCCGATACCGTCT | |
| | CTGCACGCAATACCTCCGGATTCCGTGAACAGGTCGC | |
| | TGCATGGCTGGAAGCACTCAGTGCCTCTGCGGAGCTT | |
| | CGACAGCAGTCTTTCGCTGTTGCTGCTGATGCCACTGA | |
| | GAGCTGTGAGGACCGTGTCGCGCTCACATGGAACAAT | |
| | CTCCGGGCAACCCTCCTGGTCCATCAGGCATCAGAAG | |
| | GCCTTTTCGATAATGATACCGGCGCTCTGCTCTCCCTG | |
| | GGCAGGGAAATGTTCCGCCTCGAAATTCTGGAGGATA | |
| | TTGCCCGGGATGCAGTCAGAACTCTCCATTTTGTGGAT | |
| | GAGATAGAAGTCTACCTGGCCTTCCAGACCATGCTCG | |
| | CAGAGGCACTTCAGCTCTCCACTGCCGTGGCGGAAAT | |
| | GCGTTTCTATGGCGTGTCGGGAGTGACAGCAAATGAC | |
| | CTCCGCACTGCCGAAGCCATGGTCAGAAGCCGTGAAG | |
| | AGAATGAATTTACGGACTGGTTCTCCCTCTGGGGACC | |
| | ATGGCATGCTGTACTGGCGCGTACGGAAGCTGACCGC | |
| | TGGGCGCAGGCAGAAGAGCAGGCATATGAGATGCTG | |
| | GAGAATGAGTACCCTCAGAGGGTGGCTGACCGGCTGG | |
| | CAGCATCAGGTCTGAGCGGTGATGCGGATGCGGAGAG | |
| | GGAAGCCGGTGCACAGGTGATGCGTGAGACTGAACA | |
| | GCAGATTTACCGTCAGCTGACTGACGAGGTACTGGCC | |
| | CTGCGATTGTCTGAAAACGGCTCACAACTGCACCATT | |
| | CACCTGCACATCATCATCATCATCACTAA | |
| GS2- | ATGGTTTCTTCTGTTCCGACCGCACTGGAAGTTGTTGC | 3 |
| IpaH9.8ΔLRR | TGCGACCCCGACTAGCCTGCTGATCAGCTGGGATGCT | |
| (K-free/ | CCTGCAGTTACCGTCGATCATTACTATATCACGTACGG | |
| C337A) | TGAAACCGGTCATTACTGGTACTACCAGGCATTCGCG | |
| | GTACCTGGTTCCGCGTCTACTGCTACCATCAGCGGCCT | |
| | GAGCCCGGGTGTCGACTATACCATCACTGTATACGCA | |
| | CCGTTCTCTGTTCCGGTTATGAGCCCAATCTCGATTAA | |
| | CTACCGTACCGCTAGCCTGGCTGATGCCGTGACAGCA | |
| | TGGTTCCCGGAAAACGCACAATCTGATGTATCACAGA | |
| | TATGGCATGCTTTTGAACATGAAGAGCATGCCAACAC | |
| | CTTTTCCGCGTTCCTTGACCGCCTTTCCGATACCGTCT | |
| | CTGCACGCAATACCTCCGGATTCCGTGAACAGGTCGC | |
| | TGCATGGCTGGAAGCACTCAGTGCCTCTGCGGAGCTT | |
| | CGACAGCAGTCTTTCGCTGTTGCTGCTGATGCCACTGA | |
| | GAGCGCTGAGGACCGTGTCGCGCTCACATGGAACAAT | |
| | CTCCGGGCAACCCTCCTGGTCCATCAGGCATCAGAAG | |
| | GCCTTTTCGATAATGATACCGGCGCTCTGCTCTCCCTG | |
| | GGCAGGGAAATGTTCCGCCTCGAAATTCTGGAGGATA | |
| | TTGCCCGGGATGCAGTCAGAACTCTCCATTTTGTGGAT | |
| | GAGATAGAAGTCTACCTGGCCTTCCAGACCATGCTCG | |
| | CAGAGGCACTTCAGCTCTCCACTGCCGTGGCGGAAAT | |
| | GCGTTTCTATGGCGTGTCGGGAGTGACAGCAAATGAC | |
| | CTCCGCACTGCCGAAGCCATGGTCAGAAGCCGTGAAG | |
| | AGAATGAATTTACGGACTGGTTCTCCCTCTGGGGACC | |
| | ATGGCATGCTGTACTGGCGCGTACGGAAGCTGACCGC | |
| | TGGGCGCAGGCAGAAGAGCAGGCATATGAGATGCTG | |
| | GAGAATGAGTACCCTCAGAGGGTGGCTGACCGGCTGG | |
| | CAGCATCAGGTCTGAGCGGTGATGCGGATGCGGAGAG | |
| | GGAAGCCGGTGCACAGGTGATGCGTGAGACTGAACA | |
| | GCAGATTTACCGTCAGCTGACTGACGAGGTACTGGCC | |
| | CTGCGATTGTCTGAAAACGGCTCACAACTGCACCATT | |
| | CACCTGCACATCATCATCATCATCACTAA | |
| D16- | ATGAGGACCCTGAACGAGATCTACCACTGGTTCACCA | 4 |
| CHIPΔTPR | GGATGTTCGCCTTCTTCGGCAGCGGTTCTGGCCGGCTG | |
| | AACTTCGGGGACGACATCCCCAGCGCTCTTCGAATCG | |
| | CGAAGAAGAAGCGCTGGAACAGCATTGAGGAGCGGC | |
| | GCATCCACCAGGAGAGCGAGCTGCACTCCTACCTCTC | |
| | CAGGCTCATTGCCGCGGAGCGTGAGAGGGAGCTGGA | |
| | AGAGTGCCAGCGAAACCACGAGGGTGATGAGGACGA | |
| | CAGCCACGTCCGGGCCCAGCAGGCCTGCATTGAGGCC | |
| | AAGCACGACAAGTACATGGCGGACATGGACGAGCTTT | |
| | TTTCTCAGGTGGATGAGAAGAGGAAGAAGCGAGACAT | |
| | CCCCGACTACCTGTGTGGCAAGATCAGCTTTGAGCTG | |
| | ATGCGGGAGCCGTGCATCACGCCCAGTGGCATCACCT | |
| | ACGACCGCAAGGACATCGAGGAGCACCTGCAGCGTGT | |
| | GGGTCATTTTGACCCCGTGACCCGGAGCCCCCTGACC | |
| | CAGGAACAGCTCATCCCCAACTTGGCTATGAAGGAGG | |
| | TTATTGACGCATTCATCTCTGAGAATGGCTGGGTGGA | |
| | GGATTACGTCGACGGAGCAGACTACAAGGACGATGA | |
| | CGACAAGGGACATCATCATCATCATCACTAA | |
| D16- | ATGAGGACCCTGAACGAGATCTACCACTGGTTCACCA | 5 |
| CHIPΔTPR | GGATGTTCGCCTTCTTCGGCAGCGGTTCTGGCCGGCTG | |
| (K-free) | AACTTCGGGGACGACATCCCCAGCGCTCTTCGAATCG | |
| | CGAGAAGAAGACGCTGGAACAGCATTGAGGAGCGGC | |
| | GCATCCACCAGGAGAGCGAGCTGCACTCCTACCTCTC | |
| | CAGGCTCATTGCCGCGGAGCGTGAGAGGGAGCTGGA | |
| | AGAGTGCCAGCGAAACCACGAGGGTGATGAGGACGA | |
| | CAGCCACGTCCGGGCCCAGCAGGCCTGCATTGAGGCC | |
| | AGACACGACAGATACATGGCGGACATGGACGAGCTTT | |
| | TTTCTCAGGTGGATGAGAGAAGGAGAAGACGAGACAT | |
| | CCCCGACTACCTGTGTGGCAGAATCAGCTTTGAGCTG | |
| | ATGCGGGAGCCGTGCATCACGCCCAGTGGCATCACCT | |
| | ACGACCGCAGAGACATCGAGGAGCACCTGCAGCGTGT | |
| | GGGTCATTTTGACCCCGTGACCCGGAGCCCCCTGACC | |
| | CAGGAACAGCTCATCCCCAACTTGGCTATGAGAGAGG | |
| | TTATTGACGCATTCATCTCTGAGAATGGCTGGGTGGA | |
| | GGATTACGTCGACGGAGCAGACTACAAGGACGATGA | |
| | CGACAAGGGACATCATCATCATCATCACTAA | |

### Transfection

All mammalian cells are cultured in a 37° C. static incubator with 5% CO₂. For the GS2-uAb constructs, HEK293-T cells were plated on 24-well plates at a density of 5×10⁴ cells/well 24 hours prior to transfection. On the date of transfection, 500 ng of total DNA (with a 0.1:0.4 ratio of target:uAb) was transiently transfected using a commercial transfection reagent jetPRIME (Polyplus Transfection). Media was changed 4 hours after transfection and cells were collected in 1× phosphate-buffered saline (PBS) at 24 hour-, 48 hour-, and 72 hour-time points for flow cytometric analysis. For the D16-uAb constructs, HEK293T/17 cells were used. Prior to transfection, HEK293T/17 cells were seeded in a 24-well plate with 500 μL of DMEM Media (+10% FBS, +1% PS) and at a density of 2.1×10⁶ cells per well and were allowed to grow for 24 hours in a 37° C. static incubator with 5% CO₂. To prepare plasmid DNA for transfection, 250 ng of target protein fusion and 250 ng of the construct were added to 25 μL of Opti-MEM Media and 1 μL of Enhancer reagent for each well in the experiment. Separately, 1.5 μL of Lipofectamine 3000 was added to another 25 μL of Opti-MEM for each well in the experiment. Then, each master mix was combined so that a total of 50 μL would be available for each well, which was added in a dropwise fashion. The plates were again incubated in a 37° C. static incubator with 5% CO2 for 24 hour-, 48 hour-, and 72 hour-time points.

### Flow Cytometric Analysis

After an appropriately timed incubation period, the cells were collected. First, the media was removed from each well. Then, 100 μL of 0.05% trypsin, 1 mM EDTA was added to each well before incubating for 1-2 minutes at 37° C. The 24-well plates were then forcefully hit against the countertop to dislodge the cells and checked for dislodgement by light microscopy. 900 μL of DMEM Media (+10% FBS, +1% PS) was then added to each well, before the entire 1 mL of suspended cells were removed from the well and transferred to a new Eppendorf tube. All samples are spun down at 500 rcf for 5 minutes. The supernatant was removed and the pellet was resuspended in 200 μL of 1×PBS as a wash. After another 500 rcf, 5-minute spin down, the pellet was resuspended again in 200 μL of 1×PBS before flow cytometric analysis. Flow cytometric analysis was performed using a FACSCalibur or FACSMelody instrument (BD Biosciences) as described previously (Ludwicki et al., “Broad-Spectrum Proteome Editing with an Engineered Bacterial Ubiquitin Ligase Mimic,” ACS Central Science 5(5):852-866 (2019), which is hereby incorporated by reference in its entirety). Each sample was run in triplicate and FlowJo Version 10 was used to visualize and analyze the data.

### Western Blot Analysis

Expression of the GS2-IpaH9.8ΔLRR(K-free) construct and the catalytic mutant derived thereof was analyzed by Western blot analysis of the cell lysate generated from HEK293-T cells 24 hours after transfection. Cells were washed twice with PBS and lysed with NP-40. 4×SDS loading buffer with 10% β-mercaptoethanol was added to each sample followed by boiling at 95° C. for 5 minutes prior to loading on an AnykD polyacrylamide gel. After transfer to a PVDF membrane, the blot was blocked with 5% milk in 1×TBST for 1 hour at room temperature followed by incubation in 1:5000 α-HIS-HRP (Abcam, ab18184) for 1 hour at room temperature.

### Example 1—GS2-uAb(K-Free) Promotes Enhanced Degradation of FOXP3-EGFP

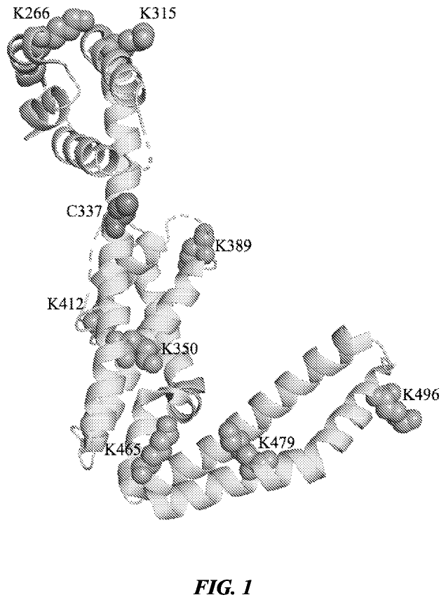

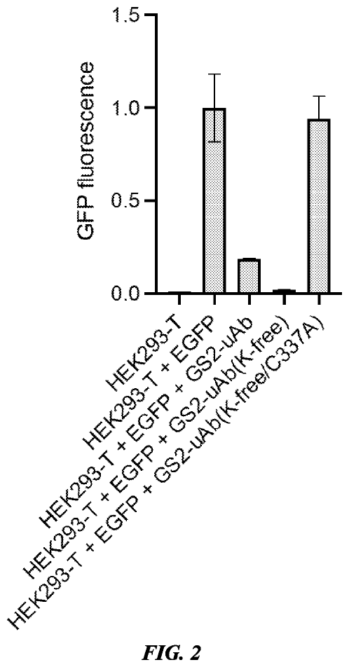

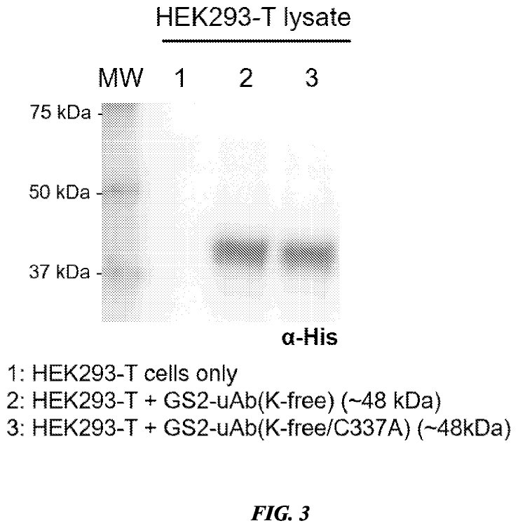

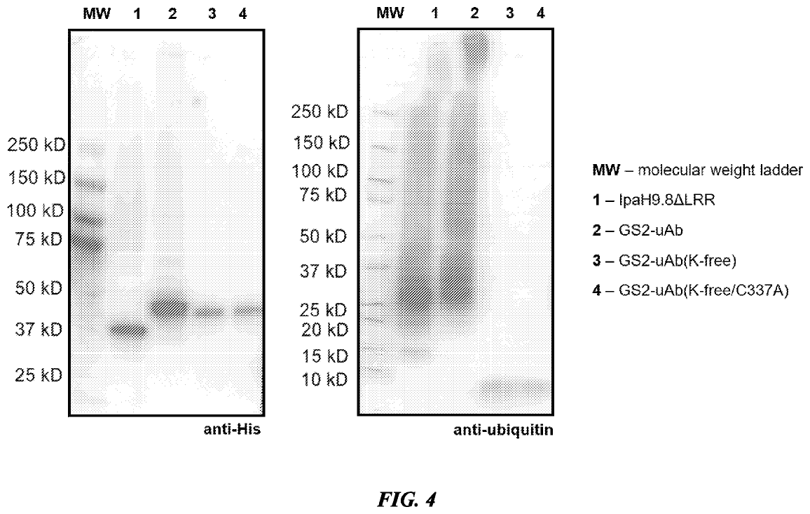

GS2-ub is a GFP-specific ubiquibody comprised of GS2 (a monobody that recognizes GFP) fused to the Shigella flexneri E3 ubiquitin ligase, IpaH9.8, lacking its native substrate-binding domain which contains 11 lysine residues (2 in the GS2 domain and 9 in the IpaH9.8ΔLRR) (FIG. 1). In HEK293-T cells expressing EGFP, the GS2-uAb construct was able to efficiently degrade the EGFP target, promoting removal of −80-90% of the intracellular EGFP as evidenced by flow cytometric analysis (FIG. 2, lane 3). When all 11 lysine residues in GS2-uAb were mutated to alanine, the resulting lysine-free ubiquibody, called GS2-uAb(K-free), was also able to promote degradation of EGFP with even greater efficiency, removing >95% of the intracellular EGFP (FIG. 2, lane 4). Degradation by the lysine-free construct was confirmed to be dependent on the catalytic domain because additional mutation of the active-site cysteine (C337A) completely abolished degradation activity of the ubiquibody (FIG. 2, lane 5). In addition to the potent target knockdown, the lysine-free ubiquibody was observed to be well expressed in the cytosol of HEK293-T cells as confirmed by Western blot analysis (FIG. 3). Moreover, Western blot analysis confirmed that substitution of all lysine residues resulted in complete elimination of autoubiquitination of the K-free ubiquibody chimera (FIG. 4, lane 3), whereas the IpaH9.8 catalytic domain alone or the original ubiquibody, each with the full complement of lysines, underwent extensive ubiquitination (FIG. 4, lanes 1 and 2).

### Example 2—D16-uAb(K-Free) Promotes Enhanced Degradation of FOXP3-EGFP

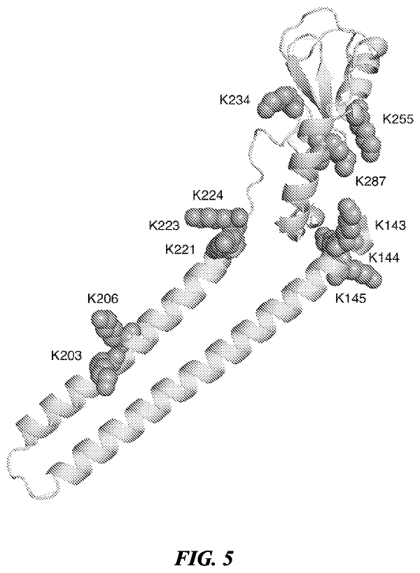

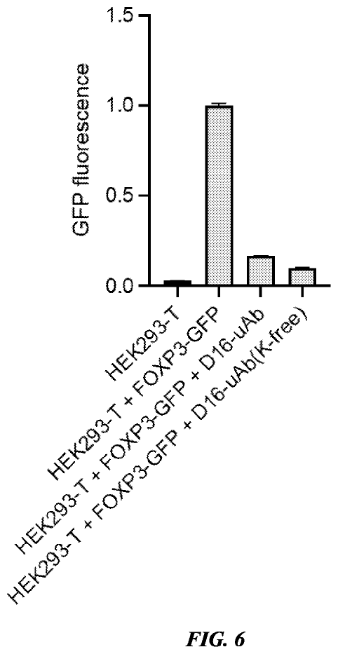

To demonstrate the broad applicability of this concept, the lysine removal strategy was extended to a second ubiquibody that involved completely different domains for substrate-binding and target ubiquitination. This second ubiquibody was comprised of D16, a short 17-residue peptide that binds specifically to human FOXP3, genetically fused to the human E3 ubiquitin ligase, CHIP, lacking its native substrate-binding domain. The resulting ubiquibody, hereafter D16-uAb, contains 11 lysines (0 in the D16 peptide and 11 in the CHIPΔTPR) (FIG. 5). In HEK293-T cells expressing FOXP3-EGFP reporter construct, the D16-uAb ubiquibody was able to efficiently degrade FOXP3-GFP, promoting removal of −80-90% of the intracellular FOXP3-EGFP as evidenced by flow cytometric analysis (FIG. 6, lane 3). When all 11 lysine residues in D16-uAb were mutated to arginine, the resulting lysine-free ubiquibody, called D16-uAb(K-free), was also able to promote degradation of FOXP3-EGFP with even greater efficiency, removing >90% of the intracellular EGFP (FIG. 6, lane 4).

### Discussion of Examples 1-2

It is well known that many E3 ubiquitin ligases in nature contain lysine residues that serve as sites for auto-ubiquitination. Such auto-ubiquitination leads to proteasomal degradation of the E3 ligase and represents a significant means by which E3 ligases auto-regulate their own stability within the cell. This native elimination mechanism could be detrimental to the long-term stability and activity of ubiquibodies, which are engineered protein chimeras that comprise a synthetic binding peptide/protein genetically fused to an E3 ubiquitin ligase lacking its native substrate-binding domain. To sidestep this possible liability, ubiquibody variants in which lysine residues were substituted with alternative amino acids such that auto-ubiquitination would no longer be possible were engineered. The results presented herein provide two representative examples and confirm that (1) the lysine removal strategy could be extended to a second, unrelated ubiquibody that targets a completely different target protein and (2) could be accomplished by mutating lysine residues to different amino acids such as alanine or

Although arginine. preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A chimeric protein molecule comprising:
(i) a degradation domain comprising an E3 ubiquitin ligase motif without lysine residues;
(ii) a targeting domain comprising a substrate-binding motif which is heterologous to the E3 ubiquitin ligase motif, and
(iii) a linker coupling said degradation domain to said targeting domain.

2. The chimeric protein molecule of claim 1, wherein said linker is heterologous to the degradation domain and the targeting domain.

3. The chimeric protein molecule of claim 1 or claim 2, wherein said substrate-binding motif and/or said linker has no lysine residues.

4. The chimeric protein molecule of any one of claims 1-3, wherein said chimeric protein molecule has increased resistance to autoubiquitination compared to a chimeric protein molecule comprising an E3 ubiquitin ligase motif having one or more lysine residues.

5. The chimeric protein molecule of any one of claims 1-4, wherein said substrate-binding motif recognizes a protein substrate and wherein said E3 ubiquitin ligase motif permits ubiquination of said protein substrate.

6. The chimeric protein molecule of any one of claims 1-5, wherein said E3 ubiquitin ligase motif possesses a cell-type specific or tissue specific ligase function.

7. The chimeric protein molecule of claim 6, wherein said ligase function is cell-type specific and the cell-type is selected from the group consisting of skin cells, muscle cells, epithelial cells, endothelial cells, stem cells, umbilical vessel cells, corneal cells, cardiomyocytes, aortic cells, corneal epithelial cells, somatic cells, fibroblasts, keratinocytes, melanocytes, adipose cells, bone cells, osteoblasts, airway cells, microvascular cells, mammary cells, vascular cells, chondrocytes, placental cells, hepatocytes, glial cells, epidermal cells, limbal stem cells, periodontal stem cells, bone marrow stromal cells, hybridoma cells, kidney cells, pancreatic islets, articular chondrocytes, neuroblasts, lymphocytes, and erythrocytes.

8. The chimeric protein molecule of any one of claims 1-7, wherein the E3 ubiquitin ligase motif is a eukaryotic E3 ligase motif.

9. The chimeric protein molecule of claim 8, wherein the eukaryotic E3 ubiquitin ligase motif is a U-box motif.

10. The chimeric protein molecule of claim 9, wherein the eukaryotic E3 ligase motif is a human Carboxyl terminus of Hsc70-Interacting Protein (“CHIP (STUB1)”) whose TPR domain located at the CHIP(STUB1)N-terminus is deleted.

11. The chimeric protein molecule of any one of claims 1-7, wherein the E3 ubiquitin ligase motif is a prokaryotic E3 ligase motif.

12. The chimeric protein molecule of claim 11, wherein the prokaryotic E3 ligase motif is from a bacterial pathogen.

13. The chimeric protein molecule of claim 12, wherein said bacterial pathogen is selected from the group consisting of Shigella, Salmonella, Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia.

14. The chimeric protein molecule of claim 12, wherein said degradation domain comprises Shigella flexneri E3 ligase, SspH1, SspH2, SlrP, AvrPtoB, LubX, N1eG5-1, NleG2-3, LegU1, LegAU13, NIeL, SopA, SidC, XopL, GobX, VirF, GALA, AnkB, or SidE.

15. The chimeric protein molecule of claim 12, wherein said degradation domain is a Shigella IpaH protein.

16. The chimeric protein molecule of claim 15, wherein said Shigella IpaH protein is selected from the group consisting of IpaH9.8, IpaH1.4, IpaH2.5, IpaH4.5, IpaH7.8, IpaH0887, IpaH1389, IpaH2022, IpaH2202, IpaH2610, and IpaH0722.

17. The chimeric protein molecule of claim 13, wherein said bacterial pathogen is Shigella flexneri.

18. The chimeric protein molecule of any one of claims 1-17, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

19. The chimeric protein molecule of claim 18, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), E13.4.3 (EGFR), EI4.2.1 (EGFR), EI4.4.2 (EGFR), E16.2.6 (EGFR), E16.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

20. The chimeric protein molecule of any one of claims 1-19, wherein said substrate-binding motif binds an intracellular protein substrate.

21. The chimeric protein molecule of any one of claims 1-19, wherein said substrate-binding motif binds a substrate selected from the group consisting of β-galactosidase, fluorescent protein, histone protein, nuclear localization signal (NLS), H-Ras protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

22. The chimeric protein molecule of any one of claims 1-19, wherein the substrate-binding motif binds a fluorescent protein selected from the group consisting of green fluorescent protein, emerald fluorescent protein, venus fluorescent protein, cerulean fluorescent protein, and enhanced cyan fluorescent protein.

23. The chimeric protein molecule of any one of claims 1-22, wherein said linker is a polypeptide linker of sufficient length to prevent the steric disruption of binding between said targeting domain and said protein substrate.

24. The chimeric protein molecule of any one of claims 1-23, wherein said linker is not cleavable.

25. The chimeric protein molecule of any one of claims 1-23, wherein said linker is enzymatically or hydrolytically cleavable.

26. A composition comprising:
the chimeric protein molecule of any one of claims 1-25; and
a pharmaceutically-acceptable carrier.

27. The composition of claim 26 further comprising:
a second agent selected from the group consisting of an anti-inflammatory agent, an antidiabetic agent, a hypolipidemic agent, a chemotherapeutic agent, an antiviral agent, an antibiotic, a metabolic agent, a small molecule inhibitor, a protein kinase inhibitor, adjuvants, apoptotic agents, a proliferative agent, and organotropic targeting agents, and any combination thereof.

28. A method of treating a disease comprising:
administering the composition of claim 26 or claim 27 to a subject having a disease, wherein the subject to whom said composition is administered has an increased expression level of said protein substrate compared to a subject not afflicted with said disease.

29. The method of claim 28, wherein said disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

30. The method of claim 28, wherein the administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

31. A method for protein substrate silencing, the method comprising:
selecting a protein substrate to be silenced;
providing a chimeric protein molecule of any one of claims 1-25; and
contacting said protein substrate and said chimeric protein molecule under conditions effective to permit the formation of a protein substrate-molecule complex, wherein said complex mediates the degradation of said protein substrate to be silenced.

32. The method of claim 31, wherein said protein substrate is selected from the group consisting of β-galactosidase, fluorescent protein, histone protein, nuclear localization signal (NLS), H-Ras protein, SHP2 protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

33. The method of claim 31, wherein the substrate is a fluorescent protein selected from the group consisting of green fluorescent protein, emerald fluorescent protein, venus fluorescent protein, cerulean fluorescent protein, and enhanced cyan fluorescent protein.

34. A method of forming a ribonucleoprotein comprising:
providing a mRNA encoding the chimeric protein molecule of any one of claims 1-25;
providing one or more polyadenosine binding proteins (“PABP”); and
assembling a ribonucleoprotein complex from the mRNA and the one or more PABPs.

35. The method of claim 34, wherein said mRNA comprises a 3′-terminal polyadenosine (poly A) tail.

36. A method of screening agents for therapeutic efficacy against a disease, said method comprising:
providing a biomolecule whose presence is mediated by a disease state;
providing a test agent comprising (i) a degradation domain comprising a E3 ubiquitin ligase motif without lysine residues, (ii) a targeting domain comprising a substrate-binding motif which is heterologous to the E3 ubiquitin ligase motif, and (iii) a linker coupling said degradation domain to said targeting domain;
contacting said biomolecule and said test agent under conditions effective for the test agent to facilitate degradation of the biomolecule;
determining the level of said biomolecule as a result of said contacting; and
identifying said test agent which, based on said determining, decreases the level of said biomolecule as being a candidate for therapeutic efficacy against said disease.

37. The method of claim 36, wherein said linker is heterologous to the degradation domain and the targeting domain.

38. The method of claim 36 or claim 37, wherein said substrate-binding motif and/or said linker has no lysine residues.

39. The method of any one of claims 36-38, wherein said test agent has increased resistance to autoubiquitination compared to a test agent comprising an E3 ubiquitin ligase motif having one or more lysine residues.

40. The method of claim 36, wherein said identifying is carried out with respect to a standard biomolecule level in a subject not afflicted with said disease.

41. The method of claim 36, wherein said identifying is carried out with respect to the biomolecule level absent said contacting.

42. The method of claim 36, wherein the method is carried out with a plurality of test agents.

43. The method of claim 36, wherein said wherein the E3 ubiquitin ligase motif is a eukaryotic E3 ligase motif.

44. The method of claim 43, wherein the eukaryotic E3 ubiquitin ligase motif is a U-box motif.

45. The method of claim 36, wherein the eukaryotic E3 ligase motif is a human Carboxyl terminus of Hsc70-Interacting Protein (“CHIP (STUB1)”) whose TPR domain located at the CHIP(STUB1)N-terminus is deleted.

46. The method of claim 36, wherein the E3 ubiquitin ligase motif is a prokaryotic E3 ligase motif.

47. The method of claim 46, wherein the prokaryotic E3 ligase motif is from a bacterial pathogen.

48. The method of claim 47, wherein said bacterial pathogen is selected from the group consisting of Shigella, Salmonella, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia.

49. The method of claim 47, wherein said degradation domain is from a bacterial pathogen and comprises Shigella flexneri E3 ligase, SspH1, SspH2, SlrP, AvrPtoB, LubX, NleG5-1, NleG2-3, LegU1, LegAU13, NIeL, SopA, SidC, XopL, GobX, VirF, GALA, AnkB, or SidE.

50. The method of claim 47, wherein said degradation domain is a Shigella IpaH protein.

51. The method of claim 50, wherein said Shigella IpaH protein is selected from the group consisting of IpaH9.8, IpaH1.4, IpaH2.5, IpaH4.5, IpaH7.8, IpaH0887, IpaH1389, IpaH2022, IpaH2202, IpaH2610, and IpaH0722.

52. The method of claim 48, wherein said bacterial pathogen is Shigella flexneri.

53. The method of claim 36, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

54. The method of claim 53, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), E13.4.3 (EGFR), E14.2.1 (EGFR), EI4.4.2 (EGFR), E16.2.6 (EGFR), E16.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

55. The method of claim 36, wherein said substrate-binding motif binds a substrate selected from the group consisting of β-galactosidase, fluorescent protein, histone protein, nuclear localization signal (NLS), H-Ras protein, SHP2 protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

56. The method of claim 36, wherein the substrate-binding motif binds a fluorescent protein selected from the group consisting of green fluorescent protein, emerald fluorescent protein, venus fluorescent protein, cerulean fluorescent protein, and enhanced cyan fluorescent protein.

57. The method of claim 36, wherein said linker is a polypeptide linker of sufficient length to prevent the steric disruption of binding between said targeting domain and said biomolecule.

58. The method of claim 36, wherein said biomolecule is associated with cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, obesity, or any combination thereof.

59. A method of screening for disease biomarkers, said method comprising:
providing a sample of diseased cells expressing one or more ligands;
providing a plurality of chimeric protein molecules comprising (i) a degradation domain comprising an E3 ubiquitin ligase motif without lysine residues, (ii) a targeting domain comprising a substrate-binding motif which is heterologous to the E3 ubiquitin ligase motif, and (iii) a linker coupling said degradation domain to said targeting domain;
contacting said sample with said plurality of chimeric protein molecules under conditions effective for the diseased cells to fail to proliferate in the absence of the chimeric protein molecule;
determining which of said chimeric protein molecules permit the diseased cells to proliferate; and
identifying, as biomarkers for the disease, based on said determining the ligands which bind to the chimeric protein molecules and permit diseased cells to proliferate.

60. The method of claim 59, wherein said linker is heterologous to the degradation domain and the targeting domain.

61. The method of claim 59 or claim 60, wherein said plurality of chimeric protein molecules and/or said linker has no lysine residues.

62. The method of any one of claims 59-61, wherein said plurality of chimeric protein molecules has increased resistance to autoubiquitination compared to a plurality of chimeric protein molecules comprising an E3 ubiquitin ligase motif having one or more lysine residues.

63. The method of claim 59, wherein said disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

64. The method of claim 59, wherein the E3 ubiquitin ligase motif is a eukaryotic E3 ligase motif.

65. The method of claim 64, wherein the eukaryotic E3 ubiquitin ligase motif is a U-box motif.

66. The method of claim 65, wherein the eukaryotic E3 ligase motif is a human Carboxyl terminus of Hsc70-Interacting Protein (“CHIP (STUB1)”) whose TPR domain located at the CHIP(STUB1)N-terminus is deleted.

67. The method of claim 59, wherein the E3 ubiquitin ligase motif is a prokaryotic E3 ligase motif.

68. The method of claim 67, wherein the prokaryotic E3 ligase motif is from a bacterial pathogen.

69. The method of claim 68, wherein said bacterial pathogen is selected from the group consisting of Shigella, Salmonella, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia.

70. The method of claim 68, wherein said degradation domain is from a bacterial pathogen and comprises Shigella flexneri E3 ligase, SspH1, SspH2, SlrP, AvrPtoB, LubX, NleG5-1, NleG2-3, LegU1, LegAU13, NIeL, SopA, SidC, XopL, GobX, VirF, GALA, AnkB, or SidE.

71. The method of claim 59, wherein said degradation domain is a Shigella IpaH protein.

72. The method of claim 71, wherein said Shigella IpaH protein is selected from the group consisting of IpaH9.8, IpaH1.4, IpaH2.5, IpaH4.5, IpaH7.8, IpaH0887, IpaH1389, IpaH2022, IpaH2202, IpaH2610, and IpaH0722.

73. The method of claim 71, wherein said bacterial pathogen is Shigella flexneri.

74. The method of claim 71, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

75. The method of claim 72, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), E13.4.3 (EGFR), E14.2.1 (EGFR), EI4.4.2 (EGFR), E16.2.6 (EGFR), E16.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2 #3 (ERα/EF), E2 #4 (ERα/EF), E2 #5 (ERα/EF), E2 #6 (ERα/EF), E2 #7 (ERα/EF), E2 #8 (ERα/EF), E2 #9 (ERα/EF), E2 #10 (ERα/EF), E2 #11 (ERα/EF), E2 #23 (ERα/EF), E3 #2 (ERα/EF), E3 #6 (ERα/EF), OHT #31 (ERα/EF), OHT #32 (ERα/EF), OHT #33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

76. An mRNA molecule encoding the chimeric protein molecule of any one of claims 1-25.

77. The mRNA molecule of claim 76, wherein said mRNA comprises a 3′-terminal polyadenosine (poly A) tail.

78. A vector encoding the RNA molecule of claim 76.

79. The vector of claim 76, wherein the vector is a viral vector.

80. The vector of claim 79, wherein the viral vector is an adenoviral vector, an adeno-associated virus vector, or a lentiviral vector.

81. An encapsulated nucleic acid molecule comprising:
(i) the mRNA molecule of claim 76 or claim 77 or the vector of any one of claims 78-80 and
(ii) a protein and/or polymer complex.

82. The encapsulated nucleic acid molecule of claim 81, wherein the nucleic acid is a mRNA molecule, wherein said mRNA molecule comprises a 3′-terminal polyadenosine (poly A) tail, and wherein said protein complex comprises one or more polyadenosine binding proteins (“PABP”).

83. The encapsulated nucleic acid molecule of claim 81, wherein said protein and/or polymer complex comprises one or more antibodies, antibody derivatives, antibody-drug conjugates, phage proteins, ubiquibodies, or combinations thereof.

84. The encapsulated nucleic acid molecule of claim 81, wherein said protein and/or polymer complex forms a nanoplex.
